(19) United States
(12) Patent Application Publication
(10) Pub. No.: US 2025/0024846 A1
(43) Pub. Date: Jan. 23, 2025

(54) DEVELOPMENT OF FOOD-GRADE FUNGAL FERMENTATES WITH ANTIMICROBIAL ACTIVITY AND THE USE THEREOF

(71) Applicant: Wisconsin Alumni Research Foundation, Madison, WI (US)

(72) Inventors: Jaehyuk YU, Madison, WI (US); Ahmad ALSHANNAQ, Madison, WI (US); Dasol CHOI, Madison, WI (US)

(21) Appl. No.: 18/714,481

(22) PCT Filed: Nov. 30, 2022
(86) PCT No.: PCT/US2022/051430

§ 371 Date: May 29, 2024

Related U.S. Application Data
(60) Provisional application No. 63/284,361, filed on Nov. 30, 2021.

(51) Int. Cl. A23L 3/3571; A01N 63/34; A01P 1/00; A23C 19/10; A61K 36/062; C12N 1/14

(52) U.S. Cl. CPC A23L 3/3571; A01N 63/34; A01P 1/00; A23C 19/10; A61K 36/062; C12N 1/14; A61K 2236/19

(57) ABSTRACT

The present invention provides antimicrobial compositions comprising an Aspergillus fermentate and methods of making said antimicrobial compositions. Also provided are products comprising the antimicrobial compositions and methods of using the antimicrobial compositions to prevent the growth of a microbe.

### CROSS-REFERENCE TO RELATED APPLICATIONS

This application claims priority to U.S. Provisional Application No. 63/284,361 filed on Nov. 30, 2021, the contents of which are incorporated by reference in their entireties.

### BACKGROUND

Foodborne illness and spoilage of food products continue to have massive negative impacts on public health and world food supplies. The Centers for Disease Control and Prevention (CDC) estimates that each year 1 in 6 Americans (about 48 million people) gets sick, 128,000 are hospitalized, and 3,000 die from eating contaminated food. The annual cost of foodborne illnesses in the United States is estimated at $15.6 billion. Listeria monocytogenes infections contribute to nearly 20% of these costs.

L. monocytogenes is a foodborne pathogen that is of major concern to the dairy and meat industries due to its ubiquitous presence and high adaptability. L. monocytogenes can grow under refrigerated and acidic conditions and is resistant to osmotic shock. L. monocytogenes is also highly tolerant to desiccation and can quickly form sanitation-resistant biofilms on equipment surfaces.

Staphylococcus aureus is another significant cause of foodborne disease that is responsible for an estimated 241,000 illnesses per year in the United States. S. aureus is notorious for its ability to become resistant to antibiotics. Infections caused by antibiotic-resistant strains often occur in epidemic waves initiated by one or a few successful clones. S. aureus releases enterotoxins that cause toxic shock-like syndromes and are implicated in food poisoning, allergic diseases, and autoimmune diseases.

Unfortunately, the misuse of antibiotics has resulted in the dramatic rise of microbes that are antibiotic resistant and resistant to food processing and preservation methods. Thus, in the absence of the discovery of new antibiotics, human mortality rates from infectious diseases are predicted to rise more than tenfold by 2050. Methicillin-resistant Staphylococcus aureus (MRSA) and Candida albicans pose a particular threat, as there are already an estimated 80,000 MRSA infections and 46,000 Candida infections every year in the United States.

Thus, there is a need in the art for antimicrobial compositions that can inhibit or prevent the growth of microbes, including microbes that cause foodborne illnesses and microbes that are antibiotic resistant.

### SUMMARY

In a first aspect, the present invention provides methods of producing an antimicrobial composition. The methods comprise (a) inoculating a medium with an Aspergillus sp. to produce an inoculated medium, (b) culturing the inoculated medium to produce an Aspergillus fermentate, and (c) filtering the Aspergillus fermentate to obtain a cell-free Aspergillus fermentate filtrate.

In some embodiments, these methods further comprise (d) extracting the cell-free Aspergillus fermentate filtrate to obtain an extract of the cell-free Aspergillus fermentate filtrate. In some embodiments, these methods further comprise (e) concentrating the extract to obtain an extract concentrate. And, in some embodiments, these methods further comprise (f) reconstituting the extract concentrate with a reconstitution solvent.

In a second aspect, the present invention provides antimicrobial compositions produced by the methods described herein. The antimicrobial compositions may have antibacterial and/or antifungal activity.

In a third aspect, the present invention provides products comprising the antimicrobial compositions described herein. In some embodiments, the products are food products or medical devices.

In a fourth aspect, the present invention provides methods of inhibiting the growth of a microbe. In some embodiments, the growth of a microbe is inhibited in or on a food product. In other embodiments, the growth of a microbe is inhibited on a surface. In other embodiments, the growth of a microbe is inhibited in a subject.

### BRIEF DESCRIPTION OF THE DRAWINGS

FIG. 1 shows a schematic depiction of the method used to produce the Aspergillus oryzae cell-free fermentates referred to herein as natural preservative 1 (NP1) and natural preservative 2 (NP2).

FIG. 2 shows bar graphs showing the diameter of zones of inhibition against Staphylococcus aureus produced by ethyl acetate extracts of NP1 and NP2. (a) Zone of inhibition produced by NP1 extracted with various organic solvents. Note that extract produced using ethyl acetate was the most effective. (b) Zone of inhibition produced by different volumes of ethyl acetate extracts of NP1 and NP2. Discs were loaded with 20, 40, 60, 80, 100, or 120 μL of the ethyl acetate extract. A disc loaded with 100 μg/mL of ampicillin was used as positive control. *, **, and *** indicate significant differences between the positive control and the samples (* p value <0.05; ** p value <0.01; *** p value <0.001). (c) Determination of the optimum conidia inoculum amount. 10⁴/mL and 10⁵/mL of A. oryzae conidia inoculum produced NP1 and NP2 fermentates with similarly sized zones of inhibition. (d) Determination of optimum culture duration. With 10⁴/mL and 10⁵/mL of A. oryzae conidia inoculum, 6 days of culture produced NP1 and NP2 fermentates with the largest zones of inhibition.

FIG. 3 shows bar graphs showing the diameter of zones of inhibition against (a) S. aureus, (b) Listeria monocytogenes, and (c) Escherichia coli produced by ethyl acetate extracts of NP1 and NP2 that were generated using Penicillium roqueforti and various Aspergillus species (i.e., A. carbonarius, A. flavus, A. fumigatus, A. oryzae, A. parasiticus, A. sojae, and A. tubingensis). Discs were loaded with 100 μL of NP1 or NP2 ethyl acetate extract. A disc loaded with 15 μg of erythromycin (a, b) or 30 μg of cefoxitin (c) was used as a positive control. Note that, while fermentates of various Aspergillus species show antimicrobial activity, fermentates of A. oryzae strains consistently display high levels of antimicrobial activity when grown in NP1 or NP2 media.

FIG. 4 shows photographs of zones of inhibition against (a) S. aureus, (b) methicillin-resistant S. aureus (MRSA), (c) L. monocytogenes, and (d) E. coli produced by ethyl acetate extracts of NP2 and NP1. Discs were loaded with 100 μL of NP2 (left column, top right disc) or NP1 (right column, top right disc) extract. Discs loaded with 5 μg ofloxacin (OFX 5, bottom left disc) or 30 μg of cefoxitin (FOX 30, bottom right disc) were used as positive controls. Methanol was used as negative control (C−, top left). Note that both the NP2 and NP1 ethyl acetate extract show antimicrobial activity that was equal to or better than that of ofloxacin and cefoxitin.

FIG. 5 shows bacterial growth curves of S. aureus (a-1), L. monocytogenes (b-1), Salmonella typhimurium (c-1), and E. coli (d-1) cultured in tryptic soy broth (TSB) made in 10× concentrated NP1 ethyl acetate extract and bacterial growth curves of S. aureus (a-2), L. monocytogenes (b-2), S. typhimurium (c-2), and E. coli (d-1) cultured in the presence of ethyl acetate extract of 10× equivalent NP2 in TSB liquid medium at 25° C. for 24-30 hours. The OD₆₀₀ was measured every 30 minutes using a Bioscreen C system. Heat-treated NP1 and NP2 were obtained by autoclaving (121° C., 50 psi) the fermentates. Methanol and ethyl acetate extracts of the fungus-free media used to produce NP1 and NP2 were used as negative controls.

FIG. 6 shows bacterial growth curves of S. aureus (a-1), L. monocytogenes (b-1), S. typhimurium (c-1), and E. coli (d-1) in TSB liquid medium generated using non-heat-treated NP1 (100%, 50%, 25%, and 10%) as a solvent as well as bacterial growth curves of S. aureus (a-2), L. monocytogenes (b-2), S. typhimurium (c-2), and E. coli (d-2) in TSB liquid medium generated using heat-treated NP2 (100%, 50%, 25%, and 10%) as a solvent at 25° C. for 24-30 hours. The OD₆₀₀ was measured every 30 minutes using a Bioscreen C system. Standard TSB medium and TSB medium generated using fungus-free NP1 or NP2 medium as a solvent were used as negative controls.

FIG. 7 shows the number of S. aureus (a), L. monocytogenes (b), S. typhimurium (c), and E. coli (d) bacteria that grew in TSB liquid medium in the presence of ethyl acetate extracts of NP1 (filled-in circle) and NP2 (filled-in square). Bacterial cultures were incubated at 37° C. for 6 hours and the numbers of live bacterial cells were measured every hour by counting colony forming units (CFU) per mL. Methanol was used as a negative control (triangle). Heat-treated NP1 and NP2 were produced by autoclaving (121° C., 50 psi) the fermentates. In all cases, 10⁵/ml of log-phase (actively growing) bacterial cells were initially used. Note that NP1 killed 100% of S. aureus cells in 1 hour, and that both NP1 and NP2 killed 100% of L. monocytogenes cells in 1-3 hours.

FIG. 8 demonstrates that cell membrane disruption is a mechanism by which NP1 and NP2 have bactericidal activity. (a) Inner membrane permeability of the Gram-positive bacteria S. aureus and L. monocytogenes were assessed based on changes in fluorescent intensity due to an interaction of Sytox Green dye with DNA released from bacterial cells (measured spectroscopically at 480 nm excitation and 522 nm emission wavelengths). (b) Outer membrane permeability of the Gram-negative bacteria S. typhimurium and E. coli were assessed based on changes in fluorescent intensity of N-phenyl-1-napthylamine (NPN) dye (measured spectroscopically at 350 nm excitation and 429 nm emission wavelengths). (c) Absorbance at 260 nm as an indicator of the amount of released cellular materials (e.g., DNA and RNA) from S. aureus, L. monocytogenes, E. coli, and S. typhimurium exposed to various treatments. The high bars represent cell rupture (death) and the release of all cellular materials. All samples were measured after 6 hours of exposure to NP1, NP2, or an ethyl acetate extract thereof. Triton-X 100 (1%) and ampicillin were used as positive controls and buffer and methanol were used as negative controls.

FIG. 9 shows cell membrane permeabilization levels of S. aureus (a), L. monocytogenes (b), S. typhimurium (c), and E. coli (d) cells treated with NP2 or NP1 extract reconstituted in methanol or methanol alone (negative control) as determined by flow cytometry experiments using the membrane-permeable dyes Syto-9 and propidium iodide (PI). The X-axis (FL3-A) shows the fluorescent intensity of the PI dye, and the Y-axis (FL1-A) shows the fluorescent intensity of the Syto-9 dye on a log scale. Highlighted regions (top right corner) show high intensity PI fluorescence, which indicates high permeability of bacterial cells. All samples were measured after 6 hours of exposure to the treatment.

FIG. 10 shows antifungal activity of NP1 and NP2 against P. roqueforti and A. fumigatus. (a) Zone of inhibition against P. roqueforti produced by 20×, 60×, and 100× concentrated NP ethyl acetate extract. Discs (13 mm) were loaded with 100 μL of the concentrated extract (bottom right disc). A disc loaded with 4 μg/mL of amphotericin B (AMB) was used as a positive control (C+). Methanol was used as negative control (C−). Note that concentrated extracts of both NP2 and NP1 show better antimicrobial activity than that of AMB. (b) Growth of A. fumigatus inoculated with inoculums of various sizes (i.e., 10⁵/mL, 10+/mL, and 10³/mL) in potato dextrose broth (PDB) liquid medium generated using various dilutions of heat-treated NP1 and NP2 (i.e., 100%, 90%, 70%, 50%, 25%, and 10%) as a solvent and cultured at 37° C. for 4 days. Standard PDB medium was used as negative control. (c) Zone of inhibition against P. roqueforti, A. fumigatus, and (′. albicans produced by 20× and 60× concentrated NP2 ethyl acetate extract obtained using NP2 at pH 8.5 (original pH) or adjusted to pH 3 or 11. Note that NP2 displayed high inhibitory activity across the tested pH range (i.e., 3-11).

FIG. 11 shows the number of A. fumigatus (a) and C. albicans (b) cells following culture in PDB liquid medium and yeast extract peptone dextrose (YPD) liquid medium, respectively, in the presence of NP2 ethyl acetate extract (open square). Approximately 5×10⁶ Aspergillus conidia and Candida cells were incubated at 37° C. for 12 hours and 30° C. for 24 hours, respectively, and the numbers of live fungal cells were counted as colony forming units (CFU) per mL. Note that NP2 killed 99.99% (10,000-fold reduction) of A. fumigatus spores and C. albicans cells in 12 hours.

FIG. 12 shows antifungal activity of NP2 against various strains of A. fumigatus and various species of Penicillium. Inhibition of growth of five different strains (i.e., AF293, F16216, F11628, CEA-10, and CEA-17) of A. fumigatus (a) and three different species (P. roqueforti, P. chrysogenuim, and P. expansum) of Penicillium (b) in potato dextrose agar (PDA) solid medium generated using various dilutions of heat-treated NP2 (i.e., 100%, 50%, 25%, and 10%) as a solvent. A. fumigatus and Penicillium species were inoculated at conidial counts of 0, 10, 100, 1,000, and 10,000 per spot and grown at 37° C. and 25° C., respectively, for 3-5 days. Standard PDA solid medium was used as negative control (PDA). Note that, while PDA made in 100% NP2 shows near-complete inhibition of Penicillium sp. growth, PDA made in as low as 10% NP2 shows high inhibition of A. fumigatus growth.

FIG. 13 shows antifungal activity of NP1 and NP2 against spores of A. fumigatus. A. fumigatus conidia (10⁵/mL) were inoculated into five different liquid media: PDB (negative control), NP2, NP1, PDB made using 100% NP2 as a solvent (NP2+PDB), and PDB made using 100% NP1 (NP1+PDB) as a solvent. The cultures were incubated at 37° C. for 24 hours. (a) Photographs of culture flasks. (b) Microscope images of fungal cells from flasks shown in (a). Note that, while NP1 shows low levels of inhibition of spore germination and hyphal growth, NP2 completely blocked spore germination, even in the presence of PDB (NP2+PDB).

FIG. 14 shows antifungal activity of NP1 and NP2 against actively growing vegetative cells of A. fumigatus. First, A. fumigatus (10⁵ conidia/mL) was grown in PDB liquid medium at 37° C. for 24 hours with shaking at 220 rpm to obtain aggregates of vegetative cells (mycelia). Then, two grams of collected mycelia was placed into flasks containing PBS (negative control), NP1, or NP2 and incubated at 37° C. for 18 hours. (a) Photographs of culture flasks. (b) Microscope images of fungal cells from flasks shown in (a). (c) Killing of A. fumigatus vegetative cells by NP2 as determined via an Alamar Blue assay. Cell viability was assessed via color change (left) and the differences in reduction (%, right) produced using four different treatments were assessed at 0, 0.5, 1, 2, 3, 4, 5, and 6 hours after inoculation. The four treatments were NP2 medium (negative control), NP2, PBS (negative control), and 1% Triton buffer (positive control for killing). (d) Photographs of plates of mycelia comprising 1 mL of each treatment that were inoculated onto PDA solid medium and incubated at 37° C. for 24 hours. Note that fungal cells treated with NP2 for 1 hour or longer did not proliferate.

FIG. 15 shows cell membrane permeabilization levels of conidia (a) and 24-hour-grown vegetative cells (b) of A. fumigatus treated with NP2 or NP1 extract reconstituted in methanol or methanol alone (negative control) as determined by flow cytometry experiments using the membrane permeable dyes Syto-9 and propidium iodide (PI). The X-axis (FL3-A) shows the fluorescent intensity of the PI dye, and the Y-axis (FL1-A) shows the fluorescent intensity of the Syto-9 dye on a log scale. Highlighted regions (top right corner) show high intensity PI fluorescence, which indicates high permeability of fungal cells. All samples were measured after 6 hours of exposure to the treatment.

FIG. 16 shows the activity of NP2 on cheeses. Three different types of commercially available sliced cheese (a) pepper jack, (b) Colby jack, and (c) cheddar were dipped into deionized (DI) sterile water (C−) or NP2, both containing 0.5% xanthan gum, for 5 seconds. Different amounts of P. roqueforti (0, 10, 100, and 1000 spores per spot) were inoculated onto the dipped cheese samples, and the cheeses were incubated at 25° C. for 2 weeks. While mold growth was visible at 3 days, 5 days, 1 week, and 2 weeks of incubation in the control groups (C−), it was effectively inhibited in the NP2-treated cheeses. (d) The same treatments were applied to cheeses that were incubated at 4° C. for 3 weeks and mold growth was checked at 2 weeks and 3 weeks of cold-room storage. Note that the NP2-treatment again blocked mold growth.

FIG. 17 shows growth curves of (a) S. aureus and (b) L. monocytogenes in TSB liquid medium and (c) (′. albicans in YPD liquid medium both generated by using individual fermentates obtained from growing A. oryzae in culture broths composed of various mixtures of the NP1/NP2 media components as described in Table 5 as a solvent (labels A-H in the legend are the media shown as A-H in Table 5). Bacterial and yeast cells were grown at 25° C. for 24 hours and OD₆₀₀ was measured every 30 minutes using a Bioscreen C system. Standard TSB medium and standard YPD liquid medium were used as negative controls for bacterial growth and fungal growth, respectively (Control). Note that the NP1 fermentates obtained from culture broths supplemented with as low as 3 g of the NP2 components per liter show high heat stability and elevated anti-bacterial and anti-fungal activity.

FIG. 18 shows growth of A. fumigatus and P. roqueforti on PDA solid medium made using heat-treated fungal fermentates as a solvent as described in FIG. 17. A. fumigatus and P. roqueforti were inoculated at conidial counts of 0, 10, 100, 1,000, and 10,000 per spot and were incubated at 37° C. and 25° C., respectively, for 3-5 days. PDA medium without NP2 was used as a negative control (PDA). Note that the culture medium made in fungal fermentate grown in a medium comprising 10-25% NP1 medium components and 75-90% NP2 medium components (see G and H in Table 5) displayed enhanced antifungal activity compared to NP1 (A) and NP2 (B) alone.

FIG. 19 demonstrates that the inclusion of casein in the fermentate medium is necessary and sufficient for NP2 antibacterial activity. Bacterial growth curves of (a) S. aureus, (b) methicillin-resistant S. aureus (MRSA), and (c) L. monocytogenes in TSB liquid medium generated using heat-treated NP2 obtained from growing A. oryzae in a media lacking one of the NP2 medium component (i.e., NP2 without casein, NP2 without glucose, NP2 without soy extract, NP2 without NaCl, and NP2 without KH₂PO₄) and containing only 10-40 g/L of pancreatic digest of casein (casein). Bacteria were grown at 25° C. for 24 hours, and OD₆₀₀ was measured every 30 minutes using a Bioscreen C system. Standard TSB medium was used as negative control (Control).

FIG. 20 shows growth of A. fumigatus and P. roqueforti inoculated at conidial counts of 0, 10, 100, 1,000, 10,000 per spot on PDA solid medium that was made using the heat-treated fungal fermentates described in FIG. 19 as a solvent. The fungi were incubated at 37° C. and 25° C., respectively, for 3-5 days. Standard PDA medium was used as a negative control (PDA). Note that the modified NP2 fermentates and the fermentates produced in casein (10-40 g/L) only media show enhanced antifungal activity against A. fumigatus and P. roqueforti.

### DETAILED DESCRIPTION

The present invention provides antimicrobial compositions comprising an Aspergillus fermentate and methods of making said antimicrobial compositions. Also provided are products comprising the antimicrobial compositions and methods of using the antimicrobial compositions to prevent the growth of a microbe.

As described in the Examples, the present inventors discovered that fermentates of Aspergillus sp. exhibit strong antimicrobial activity against bacteria and fungi. These Aspergillus fermentates offer several advantages over other antimicrobial agents. First, the fermentates were produced from a culture of a generally recognized as safe (GRAS) fungus (i.e., Aspergillus oryzae) in edible culture medium, so they are expected to be safe for use in the food, medical, and pharmaceutical industries. Second, the inventors determined that the bioactive agent(s) one of their fermentates (i.e., “NP2”) are heat stable and remain active even after being subjected to 121° C. thermal processing. Third, because the antimicrobial activity of the fermentates is strong, they can be used effectively at low levels, which may help to prevent the development of tolerance to the bioactive agent(s) in the fermentate. Fourth, because filamentous fungi offer greater metabolic versatility and secretory capacity than bacterial- and yeast-based production systems, the fermentates are expected to be easily produced at commercial scales at low cost.

### Methods of Producing Antimicrobial Compositions:

In a first aspect, the present invention provides methods of producing an antimicrobial composition. The methods comprise (a) inoculating a medium with an Aspergillus sp. to produce an inoculated medium; (b) culturing the inoculated medium to produce an Aspergillus fermentate; and (c) filtering the Aspergillus fermentate to obtain a cell-free Aspergillus fermentate filtrate.

An “antimicrobial composition” is a composition that kills or inhibits the growth of one or more microbes. A “microbe” or “microorganism” is an organism of microscopic size. Microbes include bacteria, archaea, and certain types of eukaryotes, such as microscopic fungi, protists, rotifers, and unicellular plants (e.g., algae). A microbe may be unicellular or multicellular.

The term “inoculating” refers to the act of introducing a microbe into a composition. A microbe may be introduced into a composition by simply contacting the composition with some amount of the microbe. An “Aspergillus inoculated medium” is a medium into which at least one Aspergillus sp. has been introduced. In some embodiments, the medium is inoculated with about 10⁴ to 10⁷ conidia/mL of the Aspergillus sp., preferably at least 10⁴ conidia/mL. The number of Aspergillus conidia in a substance can be determined using standard methods known in the art, including methods that utilize a hemocytometer and/or a Neubauer chamber.

The term “Aspergillus sp.” refers to any single species of the genus Aspergillus. The genus Aspergillus comprises over 800 species of conidial fungi. Examples of Aspergillus species include Aspergillus oryzae, Aspergillus terreus, Aspergillus sojae, Aspergillus nidulans, Aspergillus fumigatus, Aspergillus tubingensis, Aspergillus carbonarius, Aspergillus parasiticus, and Aspergillus flavus. In some embodiments, the Aspergillus sp. used in the method is characterized as a generally recognized as safe (GRAS) substance by the United States Food and Drug Administration (U.S. FDA). A few examples of GRAS Aspergillus sp. are Aspergillus oryzae, Aspergillus sojae, Aspergillus niger, and Aspergillus tubingensis. See the U.S. FDA inventory of GRAS notices (www.fda.gov/food/generally-recognized-safe-gras/gras-notice-inventory) for a comprehensive list of GRAS organisms. In some embodiments, the Aspergillus sp. is an A. oryzae strain selected from NRRL 3483, NRRL 6270, NRRL 2217, NRRL 32657, NRRL 694, KACC 46810, and M2014. In some embodiments, the Aspergillus sp. is an A. sojae strain selected from KATC 6375 or KACC 45026. In some embodiments, the Aspergillus sp. is an A. tubingensis strain selected from NRRL 4700 and NRRL 593. In some embodiments, the Aspergillus sp. is an A. fumigatus strain selected from F16216, CEA10, and AF293. In some embodiments, the Aspergillus sp. is the A. parasticus strain NRRL 2999. In some embodiments, the Aspergillus sp. is the A. flavus strain NRRL 3357. In some embodiments, the Aspergillus sp. is the A. carbonarius strain DTO-115-B6. In the Examples, the inventors demonstrate that the Aspergillus species A. oryzae, A. sojae, A. tubingensis, and A. fumigatus produce fermentates with antimicrobial activity. Thus, in preferred embodiments, the Aspergillus sp. is A. oryzae, A. sojae, A. tubingensis, or A. fumigatus.

As used herein, the term “culture” refers to a composition comprising at least one microbe and factors needed for the growth, reproduction, and/or replication of that microbe. Typically, these factors are provided in the form of a culture medium. (See the section below titled “Culture medium” for a detailed description of this term.) The term culture encompasses compositions into which the microbe has just been added, as well as compositions into which the microbe has been allowed to grow for a period of time.

As used herein, the term “culturing” refers to a method in which a microbe is grown in a culture. Culturing involves providing essential nutrients to the microbe and maintaining conditions (e.g., temperature, humidity, airflow, barometric pressure, percent oxygen, percent carbon dioxide) that are suitable for growth and reproduction of the microbe and then waiting for a period of time to allow the microbe to grow and reproduce (e.g., for an hour, a day, or a week). For example, in some embodiments, culturing is performed at about 22° C. to about 30° C. In some embodiments, the culturing is performed for about 2-12 days, 3-11 days, 4-10 days, or 6-10 days. In some embodiments, culturing involves agitating or shaking the culture at about 150 to 220 revolutions per minute (rpm).

As used herein, an “Aspergillus fermentate” is a product produced by culturing one or more Aspergillus species for a period of time. This composition is referred to as a “fermentate” because as Aspergillus grows in culture, it ferments the culture medium (i.e., breaks down substances in the culture medium). The Aspergillus may also secrete factors, such as carbon dioxide, ethanol, amylase, fumarate, lactic acid, citric acid, itaconic, and malate into the culture medium. Thus, the term “Aspergillus fermentate” refers to a medium that has been altered in these ways by the presence of Aspergillus.

As used herein, the term “filtering” means to pass a substance through a filter. A “filter” is a porous physical barrier that is permeable some substances but impermeable to others due to their physical size. The term “filtrate” refers to the portion of a substance that passes through the filter. In the Examples, the inventors filtered their NP1 fermentate through four layers of Miracloth to remove mycelia and then through a 0.22 or 0.45 μm polyethersulfone (PES) membrane filter unit to remove finer particles. They filtered their NP2 fermentate through four layers of Miracloth, grade GF/A glass microfiber filters, and through a 0.22 or 0.45 μm PES membrane filter unit. (Note: NP2 required an additional filtration step using a glass microfiber filter with a larger pore size (e.g., 1.6 μm) because growth of Aspergillus oryzae in tryptic soy broth (TSB) medium produces a viscous solution that would clog the more of a 0.22 or 0.45 μm PES membrane.) Thus, in some embodiments, one of more of these particular types of filters are used. However, any filter with a pore size of 0.22 μm or greater can be used to remove bacteria, yeast, and fungi from the fermentate. In some embodiments, the filtering step comprises passing the fermentate through two of more filters with different pore sizes.

In some embodiments, the filtering step is used to remove cells (i.e., mycelia) from the fermentate to produce a cell-free fermentate. As used herein, the term “cell-free” means lacking intact cells, i.e., cells in which the integrity of the plasma membrane is maintained. This is accomplished by using a filter that is impermeable to cells (i.e., comprises pores smaller than cells). The removal of living cells creates a sterile composition. Thus, in some embodiments, the filtrate is a cell-free, sterile filtrate. Alternatively, the fermentate may be sterilized via extraction or using heat (e.g., via autoclaving, boiling, or subjecting the fermentate to a temperature greater than 100° C. for at least 5 minutes).

In some embodiments, the methods further comprise (d) extracting the cell-free Aspergillus fermentate filtrate to obtain an extract of the cell-free Aspergillus fermentate filtrate.

As used herein, the term “extracting” refers to a process of selectively removing a compound of interest from a mixture. An “extract” is the product of one or more extractions. Extraction methods are known in the art and include, for example, solvent extraction, filtration, membrane separation, chromatography, precipitation, distillation, and electrophoretic methods. In the Examples, Aspergillus fermentate extracts were prepared using liquid biphasic extraction, but other methods may be used.

In some embodiments, the extracting step comprises adding an extractant to the Aspergillus fermentate. As used herein, an “extractant” is a solvent that is used to physically separate a subset of solutes from a solution via a multi-phasic liquid separation technique. Examples of extractants include acetone, ethanol, ethyl acetate, methanol, chloroform, diethyl ether, dichloromethane, methyl acetate, tetrahydrofuran, “EMW” (i.e., a mixture of ethyl acetate, methanol, and water), and “CEF” (i.e., a mixture of chloroform, ethyl acetate, and formic acid). In some embodiments, the extractant is suitable for use in a biphasic liquid extraction. In some embodiments, the extractant is a polar solvent. In some embodiments, the extractant is an aprotic polar solvent. In the Examples, the inventors used ethyl acetate to perform extractions on the cell-free Aspergillus fermentate filtrate. Thus, in some embodiments, the extractant is ethyl acetate.

In these embodiments, the fermentate-extractant mixture is allowed to separate into immiscible layers based on the different densities of the fermentate and the extractant. Then, the extractant layer is separated from the rest of the mixture as an Aspergillus fermentate extract. In some embodiments, the extractant is added to the fermentate at an extractant:fermentate ratio of about 3:1, 2:1, 1:1, 1:2, or 1:3.

In some embodiments, the methods further comprise (e) concentrating the extract to obtain an extract concentrate.

As used herein, the term “concentrating” refers to a process of removing a solvent or another diluting agent from a solution. Concentrating produces a “concentrate”, i.e., a solution comprises a higher concentration of at least one solute as compared to the original solution prior to the concentrating step. In some embodiments, the extract is concentrated via drying. An extract may be dried via dehydration, lyophilization, convection air drying, or any other method available to those skilled in the art.

Methods in which the extract is concentrated may further comprise (f) reconstituting the extract concentrate with a reconstitution solvent. The term “reconstituting” refers to a process in which a dried substance (e.g., a dried extract) is dissolved or diluted in a solvent. Accordingly, as used herein, the term “reconstitution solvent” refers to a solvent used to solubilize a dried extract. Any solvent in which the dried extract will dissolve may be utilized and should be selected based on the intended use for the antimicrobial composition. Examples of reconstitution solvents include, without limitation, methanol, ethanol, water, saline, drinkable beverages, ointments, and lotions. In the Examples, the inventors reconstituted their extract concentrates in methanol. Thus, in some embodiments, the reconstitution solvent is methanol.

In the Examples, the inventor subjected fermentates to several repeated rounds of extraction by performing an additional extraction on the reconstituted extract from the previous round of extraction. Thus, in some embodiments, the steps of (d) extracting the cell-free Aspergillus fermentate filtrate to obtain an extract of the cell-free Aspergillus fermentate filtrate, (e) concentrating the extract to obtain an extract concentrate, and (f) reconstituting the extract concentrate with a reconstitution solvent are repeated several times. For example, in some embodiments, these steps are repeated 2, 3, 4, 5, 6, 7, 8, 9, or more times. In some embodiments, the two or more rounds of extractions are performed using different extractants and/or different extraction methods. For example, the fermentate may be subjected to both a solvent-based extraction and a chromatography-based extraction.

### Culture Medium:

As used herein, a “culture medium” or “medium” is a composition designed to support the growth of a microbe. A culture medium may be a solid, liquid, or semi-solid composition. Typically, a culture medium comprises a carbon source, a nitrogen source, and micronutrients. Examples of suitable carbon sources include carbohydrates and alcohols. Examples of suitable nitrogen sources include amino acids, peptides, and ammonium salt. Examples of suitable micronutrients include trace metals, cofactors, vitamins, and essential salts. A medium may also comprise a salt used for osmolarity balance (e.g., sodium, chloride, potassium) and/or a pH buffering agent (e.g., a phosphate).

In some embodiments, the culture medium used to grow Aspergillus sp. comprises a peptone. A “peptone” or “protein hydrolysate” is a soluble mixture of polypeptides and amino acids that are formed by the partial hydrolysis of proteins. Peptones may be used in a medium as a source of protein, nitrogen, carbohydrates, and/or micronutrients. Peptones may be prepared by digesting bacteria (e.g., E. coli), fungi (e.g., yeast), plant matter (e.g., cotton, soy, wheat, or pea), or animal tissues or proteins (e.g., casein). Exemplary peptones include, without limitation, pancreatic digests of casein, papaic digests of soybean, yeast extracts, and malt extracts. A “pancreatic digest of casein” is an enzymatic digest of casein, which is a protein found in mammalian milk. A “papaic digest of soybean” is an enzymatic digest of soybean meal. A “yeast extract” is a composition comprising the cell contents of yeast without the cell walls. Typically, a yeast extract is created by heating yeast until the cells rupture and yeast enzymes begin to digest the cell contents. A “malt extract” is sugar extracted from malted grain.

In some embodiments, the culture medium used to grow Aspergillus sp. comprises a carbohydrate. A “carbohydrate” is a sugar molecule that consists of carbon, hydrogen, and oxygen, and typically has a hydrogen to oxygen ratio of 2:1. Carbohydrates include monosaccharides, disaccharides, and polysaccharides. Exemplary carbohydrates include, without limitation, dextrose and maltose.

In some embodiments, the culture medium comprises one or more micronutrients. Examples of micronutrients include minerals and vitamins. Micronutrients may be provided in the form of a salt.

In some embodiments, the culture medium comprises a pH buffering agent. A “pH buffering agent” is a weak acid or base that is used to adjust the pH of a solution or to maintain the pH of a solution following the addition of another acid or base. Non-limiting examples of pH buffering agents include phosphate buffers, acetic acid, citric acid, Tris, HEPES, MOPS, and PIPES.

In the Examples, the inventors prepared two different fermentates, referred to as “NP1” and “NP2”, using two different culture media. The medium used to prepare NP1 contained malt extract, maltose, dextrose, and yeast extract. Thus, in some embodiments, the culture medium comprises one or more of these ingredients. In some embodiments, the culture comprises, per 1 liter unit volume: 1.0-10 g of malt extract, 1-5 g of maltose, 1.0-10 g of dextrose, and 1-5 g of yeast extract. In preferred embodiments, the culture medium is the medium used to prepare the NP1 fermentate (referred to herein as “NP1 medium”). The composition of this medium is provided in Table 1.

The medium that was initially used to prepare NP2 contained pancreatic digest of casein, papaic digest of soybean, dextrose, sodium chloride, and dipotassium phosphate. However, the inventors determined that the only component of this medium that is necessary for NP2 antimicrobial activity is pancreatic digest of casein. Thus, in preferred embodiments, the culture medium comprises an enzymatic digest of casein. Specifically, in some embodiments, the culture medium comprises 10-40 g of pancreatic digest of casein per 1 liter unit volume. In some embodiments, the culture medium further comprises one or more of papaic digest of soybean, dextrose, sodium chloride, and dipotassium phosphate. Specifically, in some embodiments, the culture medium comprises 1-5 g of papaic digest of soybean, 1.0-10 g of dextrose, 1-10 g of sodium chloride, and/or 1-5 g of dipotassium phosphate per 1 liter unit volume. In some embodiments, the culture medium is the medium that was initially used to prepare NP2, which is referred to herein as “NP2 medium” (see Table 1).

In the Examples, the inventors demonstrate that fermentates prepared in a combination of NP1 medium and NP2 medium have higher and broader antibacterial and antifungal activities as compared to fermentates prepared in either NP1 medium or NP2 medium alone. Thus, in some embodiments, the culture medium is a medium comprising the components of both NP1 medium and NP2 medium. In preferred embodiments, the culture medium comprises a mixture of 10-25% NP1 medium and 75-90% NP2 medium.

In preferred embodiments, the medium comprises only food-grade ingredients. As used herein, the term “food grade” is used to refer to substances that are non-toxic and safe for consumption by humans and/or animals.

### Antimicrobial Compositions:

In a second aspect, the present invention provides antimicrobial compositions produced by the methods described herein. In some embodiments, the antimicrobial composition is food grade.

In some embodiments, the antimicrobial composition is an antibacterial composition. An “antibacterial composition” is a composition that inhibits or prevents the growth or proliferation of a bacterium and/or kills the bacterium. Antibacterial compositions may have antibacterial activity against Gram-positive bacteria and/or Gram-negative bacteria. Antibacterial compositions may have bactericidal activity and/or bacteriostatic activity. Antibacterial activity can be assessed using an assay that measures the growth, proliferation, or viability of a bacterium in a sample following treatment with an antibacterial composition. In the Examples, the inventors demonstrate that their antimicrobial compositions have antibacterial activity against Salmonella typhimurium, Escherichia coli, Staphylococcus aureus, and Listeria monocytogenes. Thus, in some embodiments, the antimicrobial compositions have antibacterial activity against one or more of these bacteria.

In some embodiments, the antimicrobial composition is an antifungal composition. An “antifungal composition” is a composition that inhibits or prevents the growth or proliferation of a fungus and/or kills the fungus. Antifungal compositions may have fungicidal activity and/or fungistatic activity. Antifungal activity can be assessed using an assay that measures the growth, proliferation, or viability a fungus in a sample following treatment with an antifungal composition. In the Examples, the inventors demonstrate their antimicrobial compositions have antifungal activity against Aspergillus flavus, Aspergillus fumigatus, Candida albicans, Candida auris, Penicillium roqueforti, Penicillium chrysogenum, and Penicillium expansum. Thus, in some embodiments, the antimicrobial compositions have antifungal activity against one or more of these fungi.

In the Examples, the inventors determine that the antimicrobial activity of the fermentate NP2 is heat stable, i.e., it is retained even after the fermentate is heated to a temperature of more than 100° C. Thus, in some embodiments, the antimicrobial activity of the antimicrobial composition is heat stable. In some embodiments, the antimicrobial composition exhibits an antimicrobial activity over a temperature range of 20 to 150° C. or over a temperature range of 20 to 130° C.

In some embodiments, the antimicrobial composition comprises one or more additives. Appropriate additives include preservatives, chelating agents, antioxidants, cryoprotective agents, stabilizers, emulsifiers, texturizers, and gelling agents. Examples of preservatives include ethylenediaminetetraacetic acid (EDTA), parabens, sodium benzoate, and sorbic acid. Examples of chelating agents include nitrilotriacetic acid, EDTA, diethylene triamine penta-acetic acid (DTPA), propylene diamine tetra-acetic acid, and ethylene diamine-N,N″-di(hydroxyphenyl or hydroxy-methylphenyl) acetic acid. Examples of antioxidants include ascorbic acid, butylated hydroxy anisole, butylated hydroxyl toluene, methionine, sodium metabisulfite, phosphoric acid, tartaric acid, and tocopherol. Examples of cryoprotectants include gelatin, glycerol, milk, mannitol, and skim milk. Examples of stabilizers and texturizers include stearic acid, palmitic acid, stearyl alcohol, cetyl alcohol, behenyl alcohol, and polyethylene glycols. Examples of emulsifiers include cetyl trimethylammonium bromide, polyvinyl alcohol, and polyvinyl pyrrolidone. Examples of gelling agents include gelatin, gums, flax, and starches, such as xanthan gum, galactomannan gum, cassia starch, and pectin.

In the Examples, the inventors used xanthan gum as a binding agent that allowed them to adhere their antimicrobial composition to the surface of cheese samples. Thus, in some embodiments, the antimicrobial compositions further comprise a food-grade binding agent. As used herein, a “food-grade binding agent” is a non-toxic substance that holds the antimicrobial composition on the surface of a food. Examples of food-grade binding agents include waxes, resins, and gelling agents. In some embodiments, the food-grade binding agent is xanthan gum.

In some embodiments, the antimicrobial composition is formulated for oral administration. For example, it may be formulated as a tablet, a capsule, a powder, a troche, a syrup, a liquid suspension, an emulsion, a solution, or a beverage. In these embodiments, the compositions may include an edible carrier. Non-limiting examples of an edible carriers include cornstarch, lactose, sucrose, bean flake, peanut oil, olive oil, sesame oil, and propylene glycol.

In other embodiments, the antimicrobial composition is formulated for topical administration. For example, it may be formulated as a paste, ointment, oil, cream, lotion, gel, tincture, powder, spray, patch, or bandage.

### Products Comprising the Antimicrobial Compositions:

The antimicrobial compositions of the present invention were designed to inhibit the growth of microbes on and in various products and organisms. Thus, in a third aspect, the present invention provides products comprising the antimicrobial compositions described herein.

In the Examples, the inventors demonstrate that their compositions protect against several foodborne pathogens. Thus, in some embodiments, the product is a food product. As used herein, a “food product” is a product that is prepared for consumption by a human or animal. Non-limiting examples of food products includes grains, breads, nuts, seeds, fruits, meat products, dairy products, candies, cookies, pizza, noodles, gums, soups, beverages, and animal feeds. In specific embodiments, the food product is a cheese.

The antimicrobial compositions may be included in the food product as a food additive that it mixed into all or a portion of the food product or it may be applied to the surface of the food product. Additional ingredients may be added along with the antimicrobial composition to improve the taste of the resulting product.

In other embodiments, the product is a medical device. A “medical device” is any device intended to be used for a medical purpose. Examples of medical devices include instruments, apparatuses, implements, machines, appliances, implants, in vitro reagents, and software intended to be used for medical purposes. In some embodiments, the medical device is a wound dressing.

### Methods for Inhibiting the Growth of Microbes:

In a fourth aspect, the present invention provides methods of inhibiting the growth or proliferation of a microbe and/or killing said microbe. The methods may be used to inhibit or kill microbes that are already present or may be used prophylactically, i.e., to prevent the growth of a microbe that is not yet present at the time the antimicrobial composition was applied or administered. Inhibiting may be measured as a reduction in growth of the microbe in the presence of the compositions provided herein relative to growth of a control, i.e., the microbe grown under the same conditions without the addition of the compositions provided herein.

Inhibition of microbe growth or proliferation can be detected using known growth inhibition assays, including zone of inhibition assays, assays that utilize the concentration, number, or optical density (e.g., absorbance at a particular wavelength) of microbes as a readout of microbial growth, and assays that detect the presence of a biomarker (e.g., aflatoxin B1) as an indicator of microbial growth. Such assays may be automated using a growth curve analysis system, such as the Bioscreen C system. Microbe killing can be detected using cell viability assays such as tetrazolium reduction assays (e.g., MTT, MTS, XTT, and WST-1), resazurin reduction assays (alamarBlue®), real-time cell viability assays (e.g., RealTime-Glo™ MT Cell Viability Assay from Promega), and assays that measure protease activity or ATP within cells as an indicator of viability.

In the present methods, the antimicrobial composition should be applied or administered in an “effective amount,” i.e., an amount sufficient to kill the microbe or to reduce, inhibit, or prevent the growth or proliferation of the microbe. An effective amount of an antimicrobial composition can be estimated initially in vitro, in cell culture assays, or in an animal model. For example, a minimum inhibitory concentration (MIC), i.e., the lowest concentration of an antimicrobial agent that inhibits the growth of a particular microbe, can be determined using a MIC assay and used as the minimal effective amount. Alternatively, a minimum bactericidal concentration (MBC) test can be used to determine the lowest amount of an antimicrobial agent that results in microbial death.

In some embodiments, the methods are used to inhibit the growth of a microbe in or on a food product. These methods comprise applying an effective amount of an antimicrobial composition described herein to the food product. In these embodiments, the antimicrobial composition is preferably food grade.

In other embodiments, the methods are used to inhibit the growth of a microbe on a surface. These methods comprise applying an effective amount of an antimicrobial composition described herein to the surface. Examples of surfaces that can be treated using the present methods include, without limitation, the surface of a medical device, the surface of a desk or bench, the surface of a food package, a food preparation surface (e.g., a surface used for cooking or food manufacturing), the surface of a plant, or the skin or fur of a human or animal.

The antimicrobial compositions may be applied to a product in several ways. For example, a product may be dipped in, coated with, or sprayed with the antimicrobial composition. In these cases, the antimicrobial composition may comprise a binding agent (e.g., xanthan gum, wax coating) to improve its ability to stick to the surface of the product. Alternatively, the antimicrobial composition may be impregnated or mixed into all or a portion of the product. The antimicrobial compositions may be applied to the product in a liquid form or may be dried and/or concentrated and applied to the product in a powdered form. In some embodiments, the antimicrobial composition is provided in the form of an antimicrobial wipe.

In still other embodiments, the methods are used to inhibit the growth of a microbe in or on a subject. These methods comprise administering an effective amount of an antimicrobial composition described herein to the subject.

In these embodiments, the methods may be used to treat or prevent a microbial infection. Examples of microbial infections include, without limitation, food poisoning, whooping cough, strep throat, ear infection, urinary tract infection, thrush, vaginitis, candidiasis, ringworm, and athlete's foot.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

As used herein, the term “administering” refers to the introduction of a substance into or onto a subject's body. Methods of administration are well known in the art and include, but are not limited to, oral administration, transdermal administration, administration by inhalation, nasal administration, topical administration, intravaginal administration, ophthalmic administration, intra-aural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instruments and human error in forming measurements, and the like.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

The present invention is based on the inventors' creation of cell-free fermentates of Aspergillus sp. that were grown in two different food-grade media. These fermentates are referred to herein as natural preservative 1 (NP1) and natural preservative 2 (NP2). In the following Example, the inventors demonstrate that NP1 and NP2 exhibit strong antimicrobial activities against both bacteria and fungi.

### Materials and Methods

Media compositions. Potato dextrose broth (PDB) contained potato starch (4 g/L) and dextrose (20 g/L). Tryptic soy broth (TSB) contained a mixture of pancreatic digest of casein (17 g/L), papaic digest of soybean (3 g/L), dextrose (2.5 g/L), sodium chloride (5 g/L), and dipotassium phosphate (2.5 g/L). yeast extract peptone dextrose (YPD) contained yeast extract (10 g/L), Bacto™ Peptone (20 g/L), and dextrose (20 g/L).

Culture of fungi for fermentate production. Aspergillus oryzae NRRL 3483 (ARS culture collection) was grown on potato dextrose agar (PDA) medium (containing 4 g potato starch, 20 g glucose, and 15 g agar in 1 L of distilled water) for 5 days at 30° C. Then, conidia (i.e., asexual spores) were harvested from the medium using sterile 0.1% Tween-80 solution. The conidia were counted using a hemocytometer and the concentration was adjusted to 10⁸ conidia/mL with sterile distilled water. Conidia suspension was stored at 4° C. and used within 2 weeks after preparation.

Composition of media for producing fermentates. Two different fermentates (i.e., NP1 and NP2) were generated using different culture conditions. The medium used to prepare NP1 contained 6.0 g malt extract, 1.8 g maltose, 6.0 g dextrose, and 1.2 g yeast extract dissolved in a final volume of 1 L of distilled water. The medium used to prepare NP2 contained 17.0 g pancreatic digest of casein, 3.0 g papaic digest of soybean, 2.5 g dextrose, 5.0 g sodium chloride, and 2.5 g dipotassium phosphate dissolved in a final volume of 1 L of distilled water. Each media was stirred for at least 20 minutes and was then sterilized under high pressure (50 psi for 20 minutes at 121° C.). The compositions of the culture media are presented in Table 1. These culture media were selected based on their ability to yield cell-free cultures with strong antimicrobial activities as compared to other fungal culture media that we tested.

TABLE 1
Compositions of NP1 medium and NP2 medium

| | |
|---|---|
| Component | Amount |

NP1 medium (1 liter)

| | | |
|---|---|---|
| Malt extract | 6 | g |
| Maltose | 1.8 | g |
| Dextrose | 6 | g |
| Yeast extract | 1.2 | g |

NP2 medium (1 liter)

| | | |
|---|---|---|
| Pancreatic digest of casein | 17 | g |
| Papaic digest of soybean | 3 | g |
| Dextrose | 2.5 | g |
| Sodium chloride | 5 | g |
| Dipotassium phosphate | 2.5 | g |

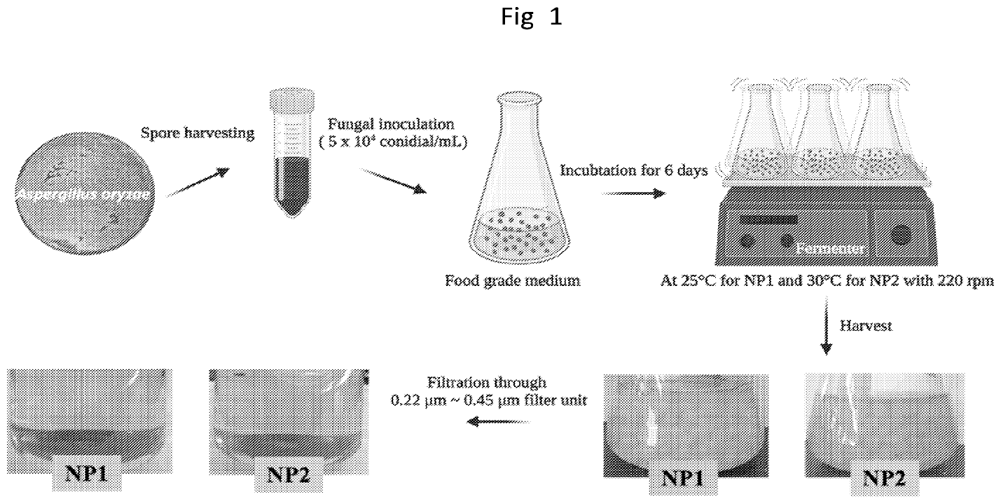

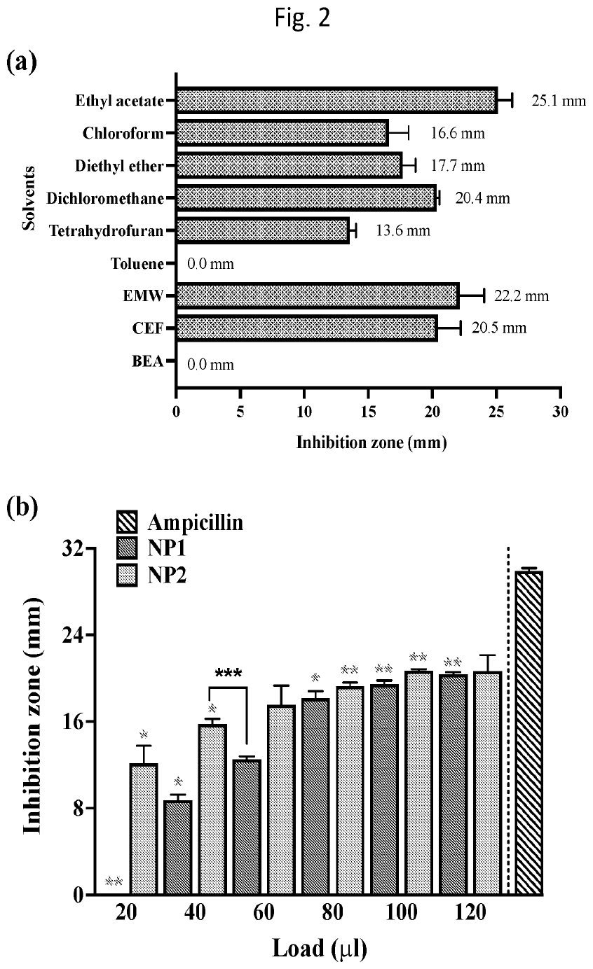

Preparation of fermentates. The process used to produce NP1 and NP2 is depicted schematically in FIG. 1. To prepare NP1, A. oryzae NRRL 3483 was inoculated, at a final concentration of 5×10⁵ conidia/mL, into Erlenmeyer flasks (250 mL) containing 150 mL of NP1 culture medium and incubated for 6 days at 25±2° C. with shaking at 220 rpm. Mycelia were separated from the culture broth via filtration through four layers of Miracloth (MilliporeSigma), and a sterile, cell-free culture fermentate (i.e., NP1) was obtained via filtration through a 0.22 μm polyethersulfone (PES) membrane filter unit (Thermo Scientific, USA).

To prepare NP2, A. oryzae NRRL 3483 was inoculated, at a final concentration of 5×10⁵ conidia/mL, into Erlenmeyer flasks (250 mL) containing 150 mL of NP2 culture medium and incubated for 6 days at 30±2° C. with shaking at 220 rpm. Mycelia were separated from the culture broth via a first filtration through four layers of Miracloth (MilliporeSigma) and a second filtration through grade GF/A glass microfiber filters (Whatman, USA). A sterile, cell-free culture fermentate (i.e., NP2) was obtained via filtration through a 0.22 μm PES membrane filter unit (Thermo Scientific, USA).

Note: While it is preferable to keep NP1 and NP2 under refrigeration (4° C.), these fermentates can be kept within a broad temperature range (e.g., at room temperature).

Extraction of fermentates. Screening of the antimicrobial activity of NP extracts produced via liquid-liquid extraction with various organic solvents was performed. The tested organic solvents (i.e., ethyl acetate, chloroform, diethyl ether, dichloromethane, tetrahydrofuran, toluene, EMW (mixture of ethyl acetate:methanol:water), CEF (mixture of chloroform:ethyl acetate:formic acid), and BEA (mixture of benzene:ethanol:ammonia hydroxide)) are described in Table 2. 20 mL of NP1 or NP2 was reacted with 20 mL of an organic solvent in 50 mL conical tubes and incubated overnight at 25±2° C. with shaking at 150 rpm. The mixtures were centrifuged at 5000 g and the organic layer was transferred to a new 50 mL conical tube. The extracts were evaporated until dry under gentle airflow. Then, the extracts were reconstituted in 1 mL of methanol and filtered with a sterile 0.45 μm filtration unit. The methanol solution was tested for antimicrobial activity.

TABLE 2
Organic solvents used to extract NP1 and NP2

| Organic solvents | | Polarity | DI |
|---|---|---|---|
| Ethyl Acetate | CH₃—COO—CH₂—CH₃ | 4.4 | 6.02 |
| Chloroform | CH₃Cl | 4.1 | 4.81 |
| Diethyl Ether | CH₃CH₂—O—CH₂CH₃ | 2.8 | 4.33 |
| Dichloromethane | CH₂Cl₂ | 3.1 | 9.1 |
| Tetrahydrofuran | C₄H₈O | 4.0 | 7.58 |
| Toluene | C₆H₅CH₃ | 2.4 | 2.38 |

| | |
|---|---|
| EMW | Ethyl acetate:Methanol:water = |
| | 10:1.35:1 (Polar, Neutral) |
| CEF | Chloroform:Ethyl acetate:Formic acid = |
| | 5:4:1 (Intermediate polarity, Acidic) |
| BEA | Benzene:Ethanol:Ammonia hydroxide = |
| | 9:1:0.1 (Nonpolar, Basic) |

*DI (dielectric constant): a measure of a solvent's polarity and the relative permittivity of a material.

Concentration of fermentates. NP1 and NP2 were extracted using ethyl acetate as described in the previous paragraph. After reconstitution, another ethyl acetate extraction was performed on 1 mL of the methanol solution. The concentrated ethyl acetate extract is 20×NP1 and NP2 fermentate equivalent. This was repeated three times, and the resulting solution is referred to as “60×” NP. The same process was also repeated 5 times, and the resulting solution is referred to as “100×” NP. The control culture filtrate was treated identically.

Preparation of bacterial inoculum. The antibacterial activity of NP1 and NP2 was tested against five bacteria: the gram-positive bacteria Listeria monocytogenes, Staphylococcus aureus, and methicillin-resistant S. aureus (MRSA), and the gram-negative bacteria Escherichia coli K12 and Salmonella typhimurium. An isolated colony was picked from the bacterial culture plate and inoculated into 9 mL of TSB liquid medium for all bacterial strains. TSB liquid medium was prepared by dissolving a mixture of pancreatic digest of casein, papaic digest of soybean, dextrose, sodium chloride, and dipotassium phosphate (30 g) in a final volume of 1,000 mL of distilled water, stirring for at least for 20 minutes, and then sterilizing under high pressure (50 psi for 20 minutes at 121° C.).

Bacterial cultures were incubated at 37° C. for 22-24 hours to produce about 10⁸ colony-forming units (cfu) per mL (cfu/mL) broth culture. The broth culture was transferred into 15 mL centrifuge tubes and spun at 5000 rpm. The supernatant was decanted, and the pellet was resuspended in 9 mL of saline. A dilution series were created by adding 1 mL of broth culture into 9 mL of saline to get a 10³ cfu/mL broth culture. The concentrations of bacteria (cfu/mL) were calculated via the spread plate technique, i.e., by pipetting 100 μL of sample from the 10³ cfu/mL and 10² cfu/mL dilution tubes onto tryptic soy agar (TSA) plates.

For long-term freezer storage of bacterial cultures, an 80% glycerol solution was prepared by diluting 100% glycerol in distilled water. Then, 750 μL of the overnight bacterial cultures was added to 250 μL of 80% glycerol to make final 20% glycerol in a 2 mL screw top tube. The prepared cultures were stored at −80° C.

Preparation of fungal inoculum. Penicillium roqueforti, Penicillium chrysogemum, Penicillium expansum (a food spoilage fungus), and Aspergillus fumigatus (a human pathogen) were used to test the antifungal activity of NP1 and NP2. To prepare an inoculum of the Penicillium sp., the Penicillium sp. were grown on PDA solid medium for 5 days at 25° C. To prepare an inoculum of A. fumigatus, A. fumigatus was grown on PDA solid medium for 3 days at 37° C. Conidia were harvested from the PDA solid medium using a sterile 0.1% Tween-80 solution. The conidia were counted using a hemocytometer and the concentration was adjusted to 10⁸ conidia/mL with sterile distilled water. The resulting conidia suspension was stored at 4° C. and was used within 2 weeks of preparation. To prepare vegetative cells containing hyphae (mycelia), a spore suspension of A. fumigatus (10⁶ conidia/ml) was inoculated into PDB liquid medium and incubated at 37° C. for 18-24 hours with shaking at 220 rpm. A. fumigatus mycelia were harvested via centrifugation at 10,000 rpm for 6 minutes, washed twice, and resuspended in PBS.

Preparation of yeast inoculum. One yeast species, i.e., Candida albicans (a human pathogen), was used to test the anti-yeast activity of NP1 and NP2. To prepare cultures of Candida albicans, single colonies were inoculated into 20 mL YPD medium and incubated at 30° C. in a rotating shaker overnight. YPD liquid medium was prepared by dissolving a mixture of yeast extract, Bacto™ Peptone, and dextrose (50 g) in a final volume of 1,000 mL of distilled water, stirring for at least for 20 minutes, and then sterilizing under high pressure (50 psi for 20 minutes at 121° C.).

Preparation of fermentate solutions for determination of antibacterial activity. To test for antibacterial activity, NP1 and NP2 were diluted in distilled water to form solutions comprising various percentages of NP1 and NP2 (i.e., 100%, 50%, 25%, and 10%). These solutions were used as a solvent in which TSB powder was dissolved to mimic the constitution of the original bacterial culture broth. See Table 3.

TABLE 3
NP solutions tested for antibacterial activity

| Dilution | Description |
|---|---|
| Control | 30 g of TSB powder dissolved in 1 L of DI water |
| 100% NP | 30 g of TSB powder dissolved in 1 L of 100% NP |
| 50% NP | 30 g of TSB powder dissolved in 1 L of 50% NP |
| | (i.e., a mixture of 500 mL DI water and 500 mL NP) |
| 25% NP | 30 g of TSB powder dissolved in 1 L of 25% NP |
| | (i.e., a mixture of 750 mL DI water and 250 mL NP) |
| 10% NP | 30 g of TSB powder dissolved in 1 L of 10% NP |
| | (i.e., a mixture of 900 mL DI water and 100 mL NP) |
| 100% NP medium | 30 g of TSB powder dissolved in 1 L of 100% NP medium |
| 50% NP medium | 30 g of TSB powder dissolved in 1 L of 50% NP medium |
| | (i.e., a mixture of 500 mL DI water and 500 mL NP medium) |
| 25% NP medium | 30 g of TSB powder dissolved in 1 L of 25% NP medium |
| | (i.e., a mixture of 750 mL DI water and 250 mL NP medium) |
| 10% NP medium | 30 g of TSB powder dissolved in 1 L of 10% NP medium |
| | (i.e., a mixture of 900 mL DI water and 100 mL NP medium) |

*NP medium refers to the culture medium used to produce NP1 or NP2 (i.e., without any fungus), whereas NP refers to the filtered fungal fermentate (i.e., NP1 or NP2).

Preparation of fermentate solutions for determination of antifungal activity. To test the antifungal activity of NP1 and NP2, NP1 and NP2 were diluted in distilled water to form solutions comprising various percentages of NP1 and NP2 (i.e., 100%, 50%, 25%, and 10%). These solutions were used as a solvent in which PDB powder was dissolved to mimic the constitution of the original fungal culture broth. See Table 4. Two different types of fungal culture were prepared: a liquid culture and a solid culture. To prepare the medium for the liquid culture, the components of PDB were dissolved in a final volume of 1 L and stirred for at least for 20 minutes. To prepare the medium for the solid culture, 15 g of agar was added to 1 L of the liquid culture medium to make potato dextrose agar (PDA) and was sterilized under high pressure (50 psi for 20 minutes at 121° C.).

TABLE 4
NP solutions tested for antifungal activity

| Dilution | Description |
|---|---|
| Control | 24 g of PDB powder dissolved in 1 L of DI water |
| 100% NP | 24 g of PDB powder dissolved in 1 L of 100% NP |
| 50% NP | 24 g of PDB powder dissolved in 1 L of 50% NP |
| | (i.e., a mixture of 500 mL DI water and 500 mL NP) |
| 25% NP | 24 g of PDB powder dissolved in 1 L of 25% NP |
| | (i.e., a mixture of 750 mL DI water and 250 mL NP) |
| 100% NP medium | 24 g of PDB powder dissolved in 1 L of 100% NP medium |
| 50% NP medium | 24 g of PDB powder dissolved in 1 L of 50% NP medium |
| | (i.e., a mixture of 500 mL DI water and 500 mL NP medium) |
| 25% NP medium | 24 g of PDB powder dissolved in 1 L of 25% NP medium |
| | (i.e., a mixture of 750 mL DI water and 250 mL NP medium) |

*NP medium refers to the culture medium used to produce NP1 or NP2 (i.e., without any fungus), whereas NP refers to the filtered fungal fermentate (i.e., NP1 or NP2).

Determination of antimicrobial activity of ethyl acetate extracts of fermentates. An agar diffusion disc test assay was performed to assess the antibacterial and antifungal activities of ethyl acetate extracts of NP1 and NP2 according to the Antimicrobial Susceptibility Testing Standards outlined by the Clinical and Laboratory Standards Institute (CLSI Mo2-A12, 2015) with some modifications.

To adjust the bacterial inoculum density for the susceptibility test, 1-2 colonies of the bacterial strains were inoculated in 9 mL TSB in 15 mL conical tubes. The turbidity of the bacterial suspensions was adjusted with TSB to a 0.5 McFarland standard reference. An absorbance of 0.08-0.1 at 600 nm demonstrated that the bacterial suspensions had an optical density of about 1 to 2×10⁸ cfu/mL. A cotton swab was used to spread the bacterial suspensions evenly on TSA plates using the spread plate technique. Then, 100 μL of ethyl acetate extract, which was reconstituted in methanol, was loaded onto 6 mm sterile paper discs. The paper discs were dried under the fume hood and placed on the surface of the TSA plates where the bacterial suspensions were spread. The plates were inverted and incubated at 37° C. for 18-22 hours. Antibacterial activity was determined by measuring the diameter of the inhibition zone around the 6 mm paper discs.

To assess the bacterial growth following treatment with the NP ethyl acetate extracts, 100 μL of ethyl acetate extract (reconstituted in methanol) was transferred into a 96-well plate and dried under a fume hood under sterile conditions. Then, 180 μL of TSB was added to the 96-well plates. After that, 20 μL of bacterial cultures (˜10³ cfu/mL, obtained via serial dilution of overnight bacterial cultures) was added to the 96-well plates. The plate was then placed in a Bioscreen C device and the absorbance at 600 nm was measured at 25° C. and 37° C. for 24-30 hours. Methanol and ethyl acetate extracts of fungus-free NP1 medium and NP2 medium were used as negative controls.

For fungal spore samples of Penicillium sp. and A. fumigatus, concentrations were adjusted to 10⁴-10⁷ conidia/mL with sterile distilled water and conidia were counted using a hemocytometer. Then, 100 μL of ethyl acetate extract was loaded onto 13 mm sterile paper discs. The paper discs were dried under a fume hood and placed on the surface of the PDA plates where the fungal spore suspensions were spread. The plates were inverted and incubated at 25° C. and 37° C. for Penicillium sp. and A. fumigatus, respectively, for 4-6 days. Antifungal activity was determined by measuring the diameter of the inhibition zone around the 13 mm paper discs. Amphotericin B (AMB; final concentration of 4 μg/mL) was used as positive control for both fungal strains.

Determination of antimicrobial activity of fermentates. To investigate the in vitro antibacterial and antifungal activity of NP1 and NP2 filtered fermentate, various TSB- and PDB-based media were prepared, as described in Table 3 and Table 4 above.

To assess bacterial growth, 180 μL of two controls (i.e., standard TSB and various dilutions of fungus-free NP medium) and each PDB liquid medium prepared in diluted NP fermentate were added to 96-well plates. Then, 20 μL of bacterial culture (˜10³ cfu/mL), which was obtained by serial dilution of overnight bacterial cultures, was added. The plate was placed in a Bioscreen C device and the absorbance at 600 nm was measured at 25° C. and 37° C. for 24-30 hours.

For screening of the antifungal activity of NP1 and NP2 against A. fumigatus, various amounts of conidia inoculum (i.e., 10⁵/mL, 10⁴/mL, and 10³/mL of A. fumigatus in 2 mL PDB liquid medium) were inoculated into various dilutions of to heat-treated NP1 and grown at 37° C. for 4 days. Standard PDB liquid medium was used as negative control. For solid culture on PDA medium, 10 μL of spore suspensions of several fungi strains (A. fumigatus strains AF293, F16216, F11628, CEA-10, and CEA-17 and the Penicillium species P. roqueforti, P. chrysogemum, P. expansum (Pe21), and P. expansum (R19)) were spread onto PDA plates made from the various PDA media described in Table 4 at final concentrations of 10, 10², 10³, and 10⁴ spores per spot. A. fumigatus was incubated at 37° C. for 4-5 days and Penicillium sp. were grown at 25° C. for 4-5 days.

To test the antifungal activity of NP1 and NP2 against spores of A. fumigatus, 10⁵ conidia/mL of A. fumigatus was inoculated into five different liquid media: PDB (negative control), NP2, NP1, PDB made in 100% NP2 (NP2+PDB), and PDB made in 100% NP1 (NP1+PDB) and incubated at 37° C. with shaking at 220 rpm for 18 hours.

To examine the fungicidal of NP2 against vegetative cells of A. fumigatus (prepared as described above), an Alamar Blue assay was performed. The Alamar Blue assay is a cell viability assay that uses the natural reducing power of living cells to convert the dye resazurin to the fluorescent molecule resorufin. In this assay, blue and low fluorescence indicate dead cells, whereas bright red fluorescence indicates living cells. Alamar Blue reduction may signify an impairment of cellular metabolism and is not necessarily specific to interruption of electron transport and mitochondrial dysfunction.

Determination of the mode of action of fermentates. We hypothesized that the mechanism of action of NP1 and NP2 might involve disrupting the membrane and structure of Gram-negative and Gram-positive bacteria. Therefore, outer membrane and inner membrane integrity assays were performed using an electron fluorescence spectrometer with specific fluorescent dyes. Samples and bacterial cells were prepared as described above in the section titled “Determination of antibacterial and antifungal activity of ethyl acetate extracts of NP1 and NP2.”

To determine the inner membrane permeability for Gram-positive bacteria, a SYTOX® Green influx assay was used. SYTOX® Green is a nucleic acid stain that penetrates cells with a compromised plasma membrane but will not penetrate the membranes of live cells. The excitation and emission wavelengths of SYTOX® Green were measured at 480 and 522 nm, respectively. 20 μL of 5 μM SYTOX® Green was added to the prepared wells. The excitation of fluorescence increased if the plasma membrane was disrupted by treatment with the ethyl acetate extract of NP1 or NP2.

For the outer membrane of Gram-negative bacteria, permeability was determined using an NPN assay. The lipophilic dye N-phenyl-1-napthylamine (NPN) is a hydrophobic fluorescent probe that fluoresces when it enters the outer membrane, i.e., the hydrophobic core of the lipid bilayer. Excitation and emission wavelengths were set at 350 and 429 nm, respectively. Samples were mixed with 20 μL of NPN solution (final concentration: 10 μM).

If the bacteria membrane is compromised, the release of the cytoplasmic contents of the cell can be monitored. By detecting absorbance at 260 nm, one can estimate the amount of DNA and RNA released from the cytoplasm. After 6 hours of incubation, the supernatants of bacterial cell suspensions were collected and the optical density at 260 nm was recorded.

To confirm all experiments regarding cell membrane permeability described above, flow cytometry with was used to determine whether ethyl acetate extract of NP1 and NP2 could damage the cytoplasmic membrane and allow the stains propidium iodide (PI) dye and SYTO 9 to penetrate and interact with intracellular nucleic acids, which causes them to become fluorescent. PI is a red-fluorescent dye that is non-permeable to intact plasma membranes and cannot enter viable cells, whereas SYTO 9 is a green-fluorescent dye that can enter both live and dead bacterial cells. After 6 hours of incubation of samples, SYTO-9 and PI stains were added, at 7.5 μM and 30 μM, respectively.

In addition to bacterial membrane disruption, fungal membrane disruption was examined by flow cytometry using the same procedure described above. Antifungal agents can disrupt membranes in two ways: (a) they can cross the cell membrane, leading to pore formation and specifically targeting B-glucan or chitin synthesis, and (b) they can interact with the cell membrane and cause cell lysis. Two types of cells of A. fumigatus were tested: conidia and 24-hour grown vegetative cells. The preparation of these two types of cells is described above in the section titled “Preparation of inoculum fungal cultures”.

Application of fermentates to cheeses. Cheeses were used to evaluate the ability of NP2 to inhibit mold growth on food products. De-ionized (DI) water was used as a negative control. Sliced version of three cheeses (i.e., pepper jack, Colby jack, and cheddar) were tested. A binding agent, 0.5% (w/w) xanthan gum, was added into the DI water and NP2 to allow them to stick to the cheeses. Cheeses were dipped into the prepared DI water and NP2 solutions for 5 seconds. The dipped cheese samples were air-dried to remove moisture. Spore suspensions of P. roqueforti containing 0 (control), 10, 100, or 1000 spores were prepared in DI water. The spore suspensions were used to inoculate the prepared cheeses (per 10 μL) in triplicate. The cheese samples were placed into petri-dishes (15 cm diameter), covered with a lid, and sealed inside a plastic bag to prevent the cheese samples from drying out. The cheese samples were incubated at 4° C. (cold room) or at 25° C. (room temperature).

Determination of the optimum combination of NP1 medium and NP2 medium. NP1 and NP2 media were generated as described in Table 1. Namely, 15 g/L of the NP1 medium components (i.e., malt extract, maltose, dextrose, and yeast extract) were used to make NP1 medium, and 30 g/L of the NP2 medium components (i.e., pancreatic digest of casein, papaic digest of soybean, dextrose, sodium chloride, and dipotassium phosphate) were used to make NP2 medium. In addition to these “standard” NP media, several NP1/NP2 combination media comprising mixtures of various amounts of the NP1 medium components and the NP2 medium components were prepared, as outlined in Table 5. To prepare fermentates in the NP1/NP2 combination media, A. oryzae was grown in these media as described above.

TABLE 5
NP1/NP2 combination media

| | | |
|---|---|---|
| | Concentration of NP1 | Concentration of NP2 |
| Medium | medium components | medium components |

| | | | | |
|---|---|---|---|---|
| A (NP1) | 15 | g/L | 0 | g/L |
| B (NP2) | 0 | g/L | 30 | g/L |
| C | 15 | g/L | 30 | g/L |
| D | 13.5 | g/L | 3 | g/L |
| E | 11.25 | g/L | 7.5 | g/L |
| F | 7.5 | g/L | 15 | g/L |
| G | 3.75 | g/L | 22.5 | g/L |
| H | 1.5 | g/L | 27 | g/L |

Identification of essential components in NP2 medium. To determine which components of the NP2 medium are essential for the antimicrobial activity of NP2, A. oryzae fungal fermentates were prepared in medium lacking single NP2 medium components, i.e., NP2 medium without pancreatic digests of casein, NP2 medium without papaic digest of soybean, NP2 medium without dextrose, NP2 medium without sodium chloride, and NP2 medium without dipotassium phosphate. Additionally, media containing only 10-40 g/L of pancreatic digest of casein (also known as tryptone) were prepared.

### Results

### Optimization of Fermentate Extracts

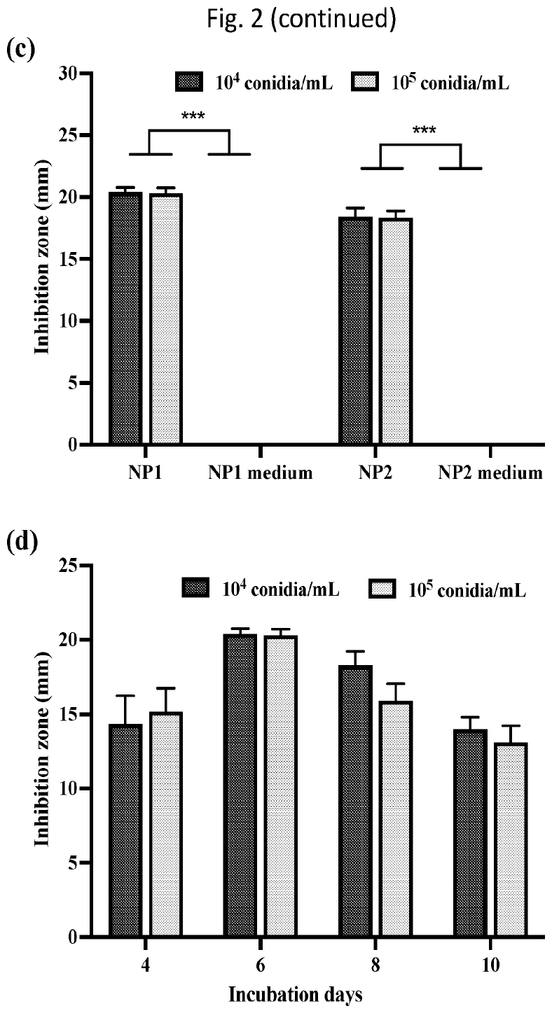

In addition to ethyl acetate, several other organic solvents were used to extract antimicrobial compounds from NP1 and NP2. These other organic solvents, which were selected based on their polarity, included chloroform, diethyl ether, dichloromethane, tetrahydrofuran, toluene, a mixture of ethyl acetate, methanol, and water (EMW), a mixture of chloroform, ethyl acetate, and formic acid (CEF), and a mixture of benzene, ethanol, and ammonia hydroxide (BEA). However, ethyl acetate (which extracts both polar and nonpolar compounds) worked better as an organic solvent for extracting antimicrobial compounds than the other tested organic solvents. For example, the ethyl acetate extract resulted in a 25.1 mm inhibition zone against S. aureus, whereas both the toluene and BEA extracts resulted in a 0 mm inhibition zone (FIG. 2a). This suggests that non-polar compounds are not solely responsible for the antimicrobial activity of the NP extracts (FIG. 2a).

To determine the dose response to the ethyl acetate extracts of NP1 and NP2, various volumes of the extracts (i.e., 20, 40, 60, 80, 100, and 120 μL) were loaded onto 6 mm paper discs and an antimicrobial susceptibility test was performed against S. aureus. The inhibition zones produced by 100 μL of extract were about 20 mm, and there was no significant difference between the 100 μL and 120 μL loading volumes (FIG. 2b). Therefore, 100 μL was selected as the loading volume for further antimicrobial tests. For comparison, the positive control, 100 μg/mL of ampicillin, resulted in an inhibition zone of about 30 mm (FIG. 2b). The media used for culturing NP1 and NP2 (i.e., NP1 medium and NP2 medium) showed no antimicrobial activity (i.e., a 0 mm inhibition zone) against S. aureus.

As shown in FIG. 2c, use of different concentrations of A. oryzae conidia did not impact the measured antimicrobial activity of the ethyl acetate extracts of NP1 and NP2. However, as shown in FIG. 2d, the culture duration did affect the antimicrobial activity of the ethyl acetate extracts. Specifically, ethyl acetate extracts of NP2 and NP1 that had been cultured for 6 days at 25-30° C. exhibited greater antimicrobial activity as compared to extracts of fermentates that were cultured for 4, 8, or 10 days.

### Antimicrobial Activity of Fermentates Generated Using Various Fungi

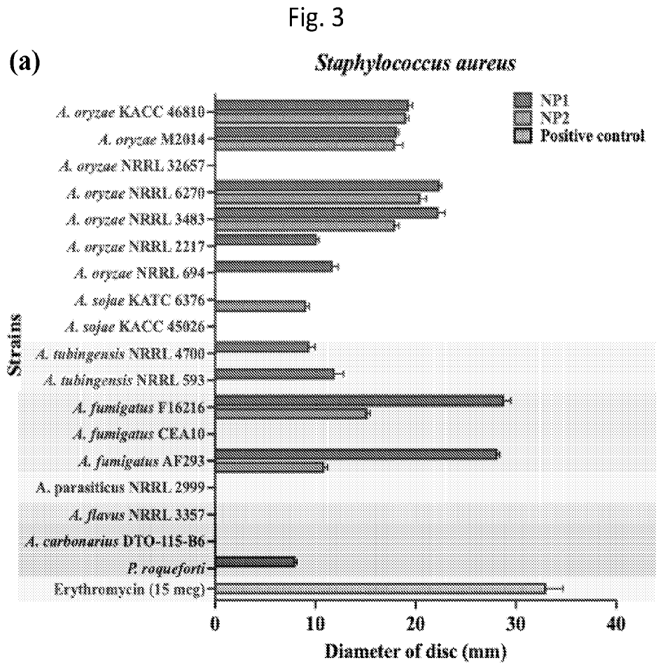

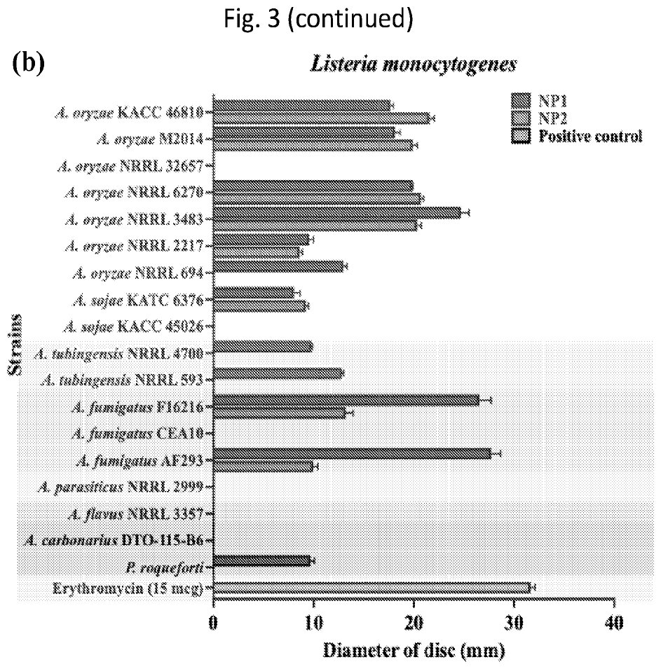

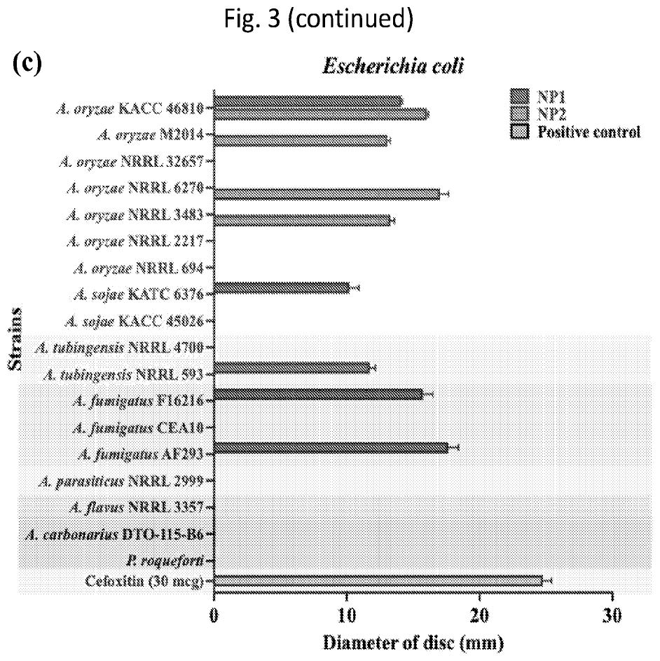

Different species and strains of Aspergillus and Penicillium were used to produce fermentates in NP1 and NP2 media. P. roqueforti and the Aspergillus species A. carbonarius, A. flavus, A. fumigatus, A. oryzae, A. parasiticus, A. sojae, and A. tubingensis were tested. Ethyl acetate extracts of these fermentates were tested against S. aureus (FIG. 3a), L. monocytogenes (FIG. 3b), and E. coli (FIG. 3c). Discs were loaded with 100 μL of the ethyl acetate extracts. Cefoxitin (30 μg) and erythromycin (15 μg) were used as positive controls. While filtered fermentates of the various fungi showed some antimicrobial activity, the fermentates made using strains of A. oryzae most consistently displayed high antimicrobial activity.

### Antibacterial Activity of Fermentates

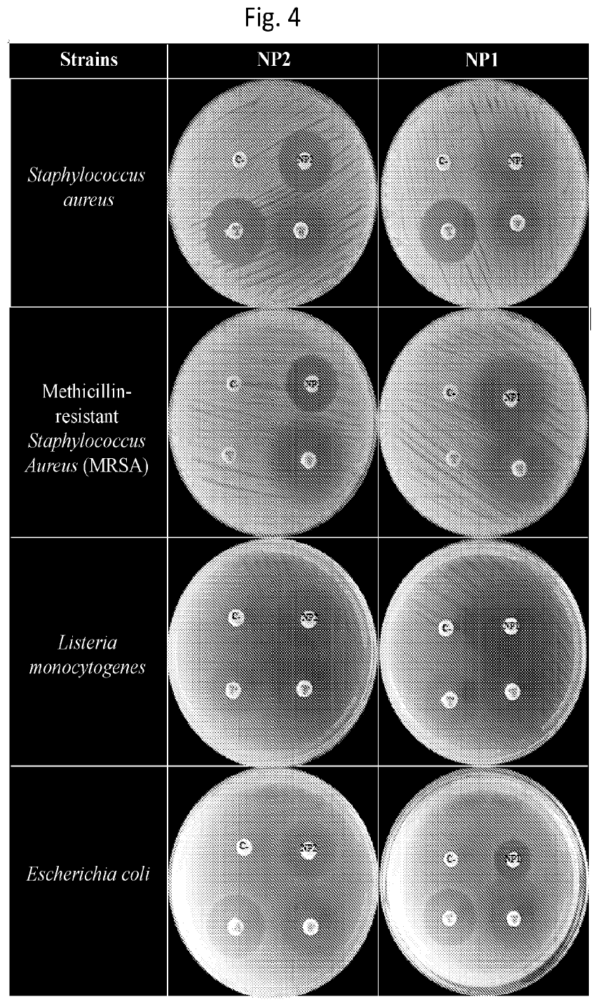

The antibacterial activity of NP1 and NP2 was tested against both Gram-positive (L. monocytogenes, S. aureus, MRSA) and Gram-negative (E. coli and S. typhimurium) bacteria. All the tested bacterial strains are foodborne pathogens. Ethyl acetate extracts of NP2 and NP1 produced large zones of inhibition against S. aureus, MRSA, L. monocytogenes, and E. coli, and both NP2 and NP1 show antimicrobial activity that is equal to or better than that of 5 μg of ofloxacin or 30 μg of cefoxitin (FIG. 4).

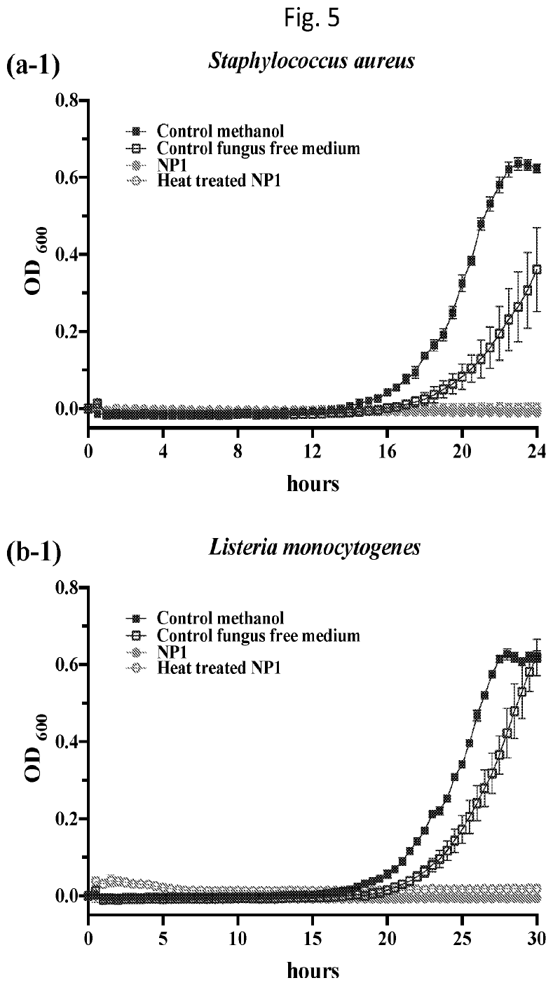

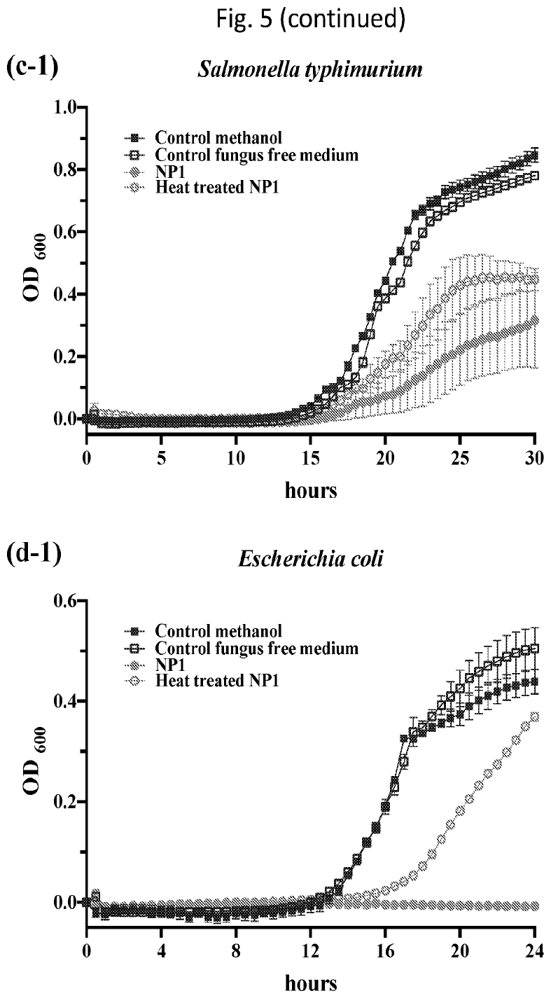

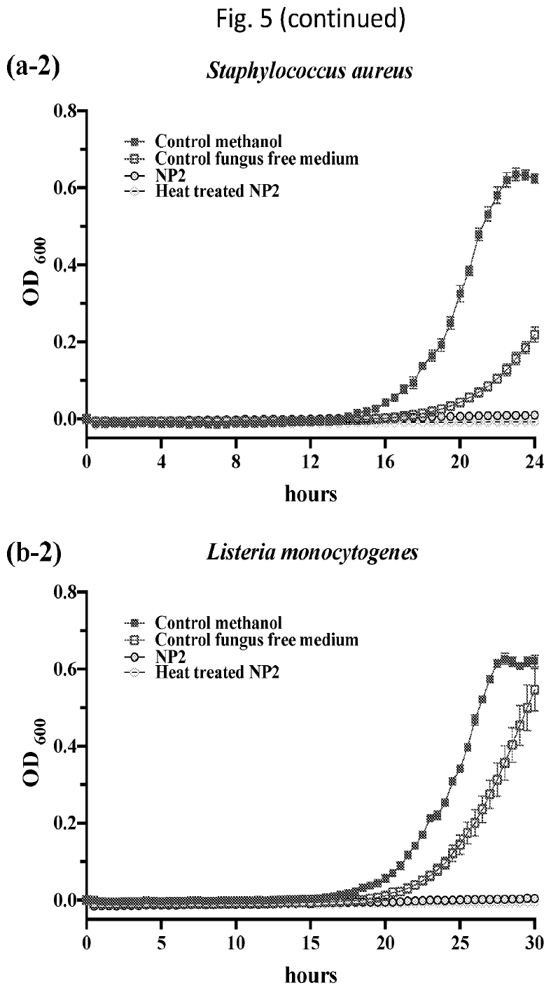

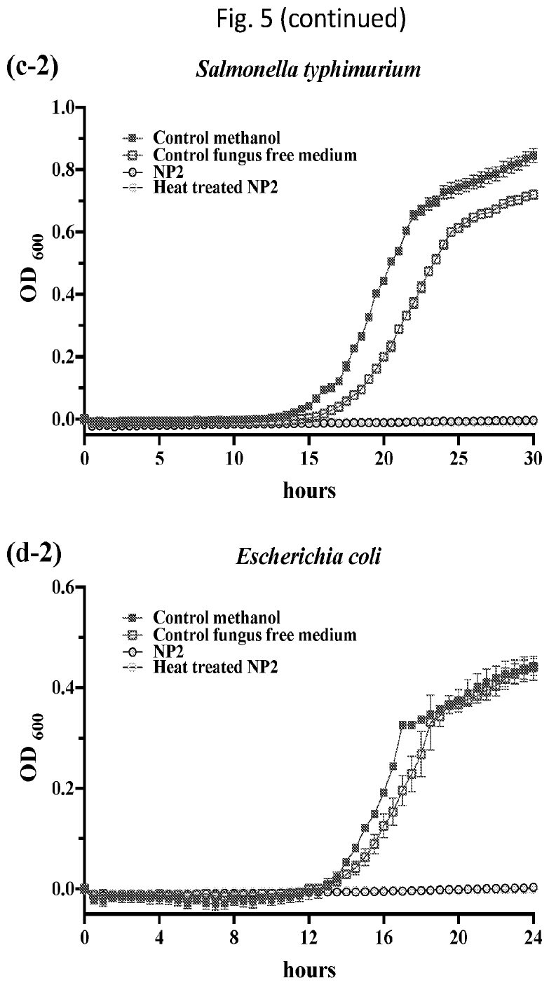

Ethyl acetate extract of 10× concentrated NP1 in TSB liquid medium showed superior antimicrobial activity against Gram-positive bacteria as compared to Gram-negative bacteria (FIGS. 5a-1, b-1, c-1, and d-1). Ethyl acetate extract of 10× concentrated NP2 in TSB liquid medium showed strong antibacterial activity against both Gram-positive and Gram-negative bacteria (FIGS. 5a-2, b-2, c-2, and d-2). While the antimicrobial compound(s) present in NP1 that are effective against Gram-negative bacteria appear to be heat-labile, the antimicrobial compound(s) in NP2 appear to be heat-resistant (FIG. 5).

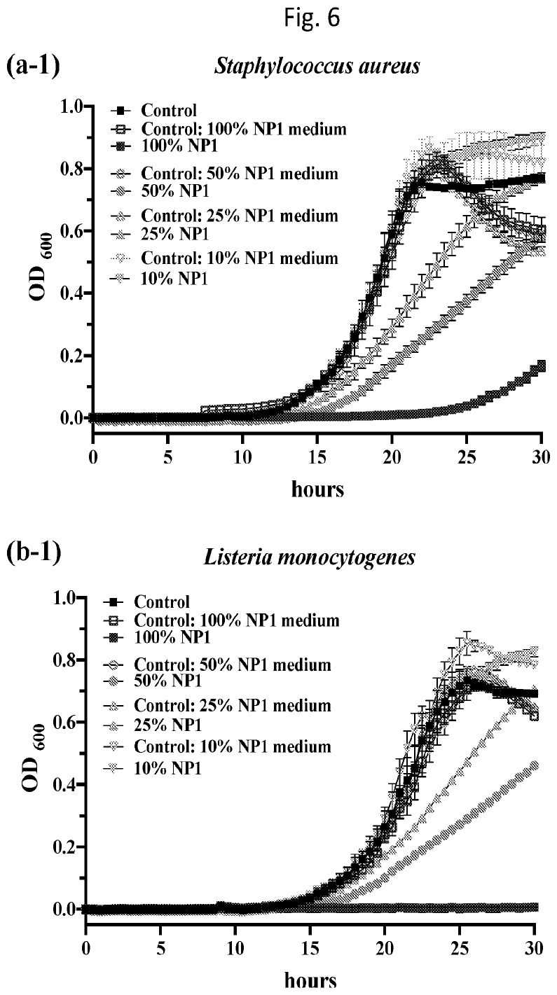

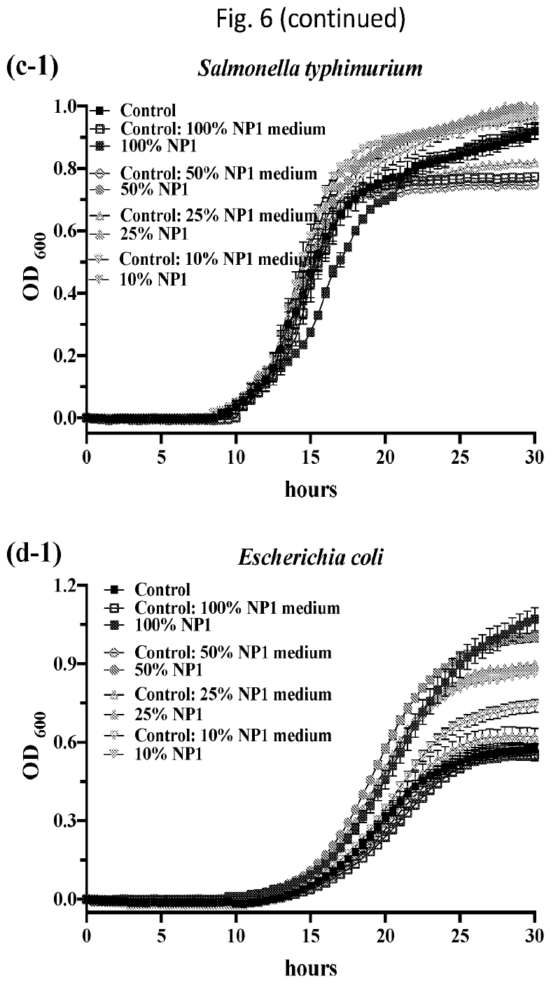

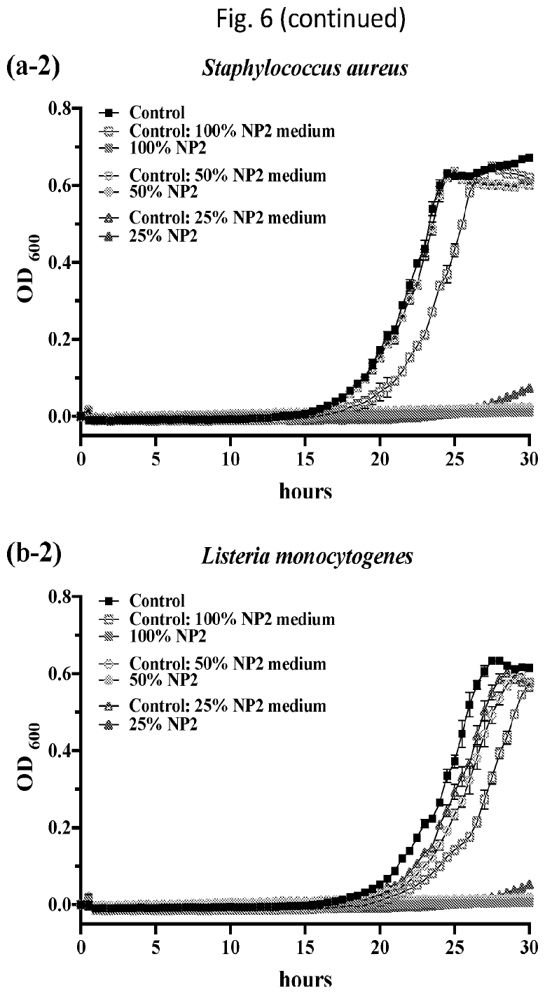

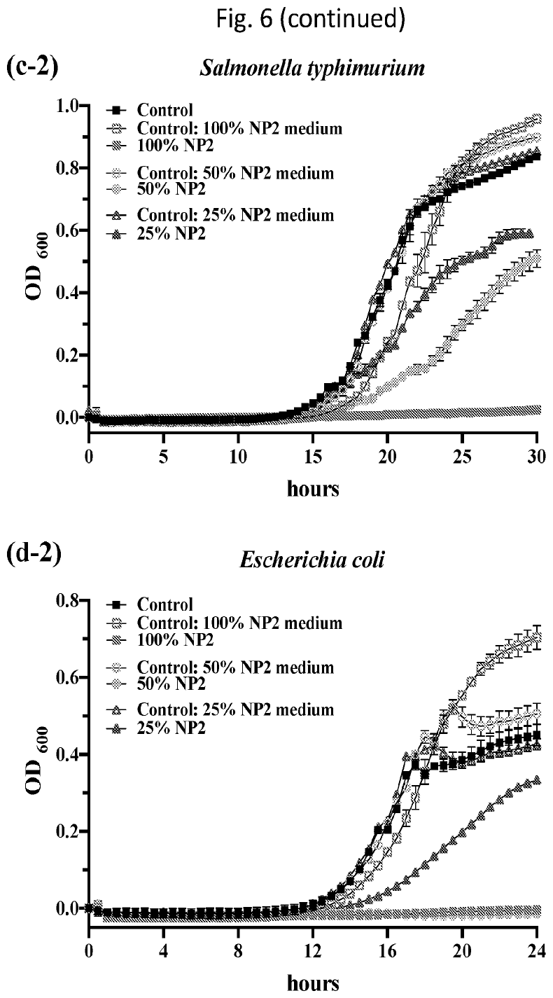

Non-heat-treated NP1 in TSB liquid medium delayed the growth of S. aureus about 20 hours (FIG. 6a-1) and inhibited the growth of L. monocytogenes (FIG. 6b-1). However, regardless of heat treatment, NP1 was unable to prevent the growth of the Gram-negative bacteria S. typhimurium and E. coli (FIGS. 6c-1 and d-1). Up to 50% TSB liquid medium generated using heat-treated NP2 prevented the growth of the Gram-positive bacteria until at least 30 hours at 25° C. (FIGS. 6a-2 and b-2). Unlike NP1, NP2 has antibacterial activity against the Gram-negative bacteria S. typhimurium and E. coli. 100% NP2 blocked the growth of S. typhimurium and up to 50% NP2 inhibited the growth of E. coli (FIGS. 6c-2 and d-2).

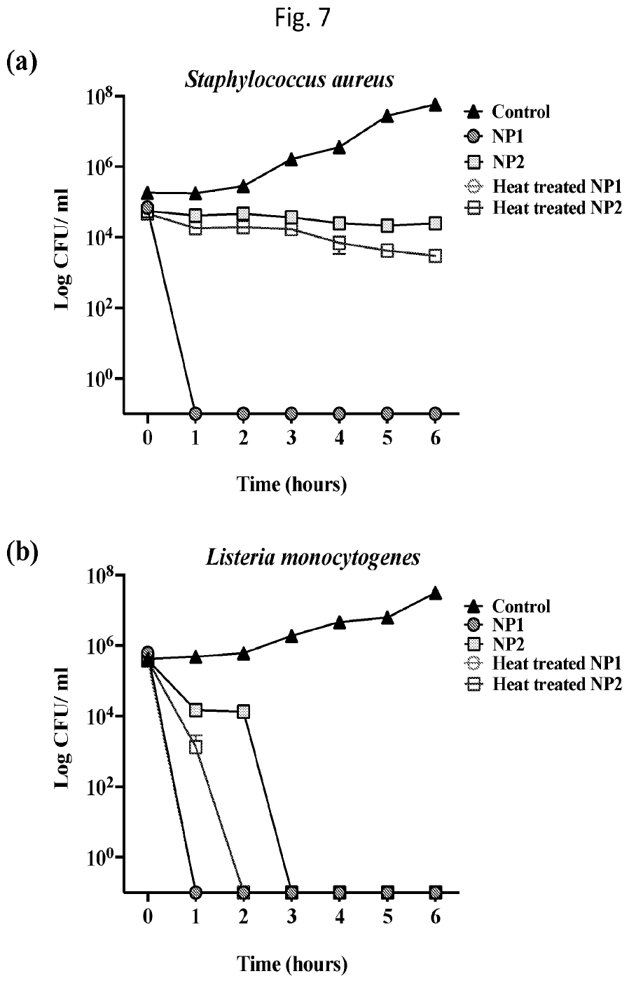

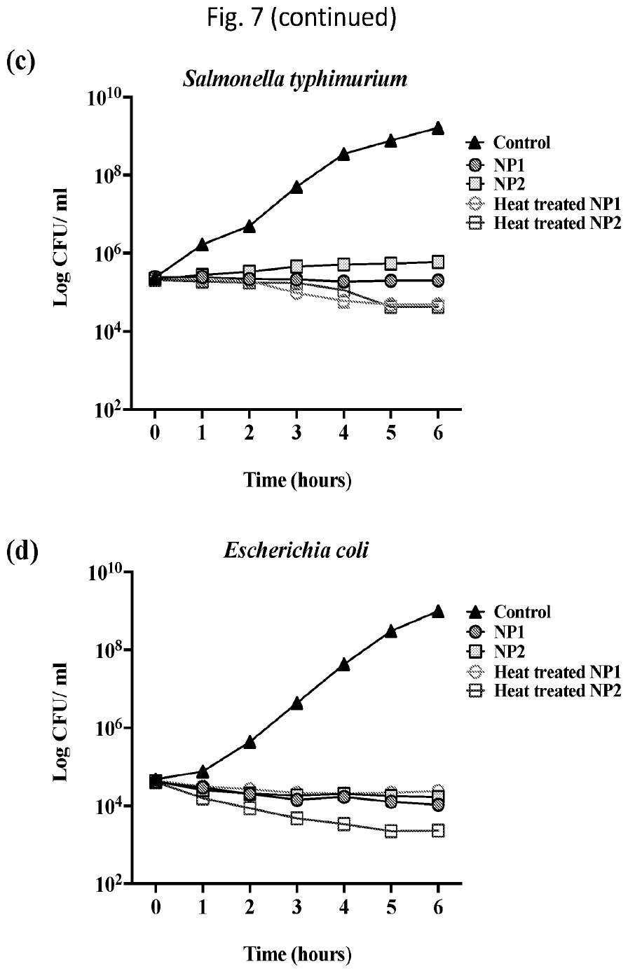

Dramatically, ethyl acetate extracts of NP1 resulted in a 100% reduction of S. aureus growth in TSB liquid medium after 1 hour (FIG. 7a), and both NP1 and NP2 can effectively kill 100% of L. monocytogenes cells in 1-3 hours (FIG. 7b). Further, both NP1 and NP2 functioned as bacteriostatic agents against log phase E. coli and S. typhimurium (FIGS. 7c and d).

### Mechanism of the Bactericidal Activity

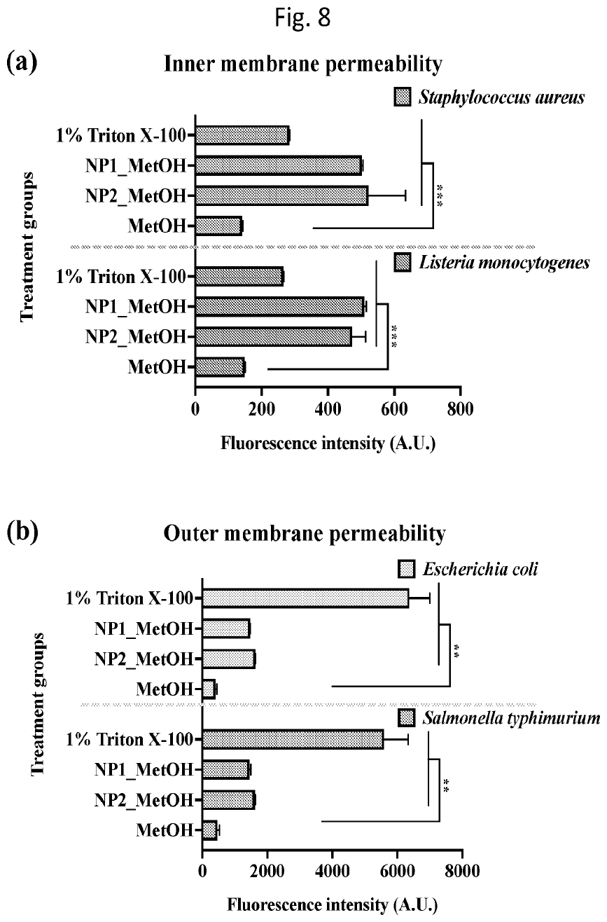

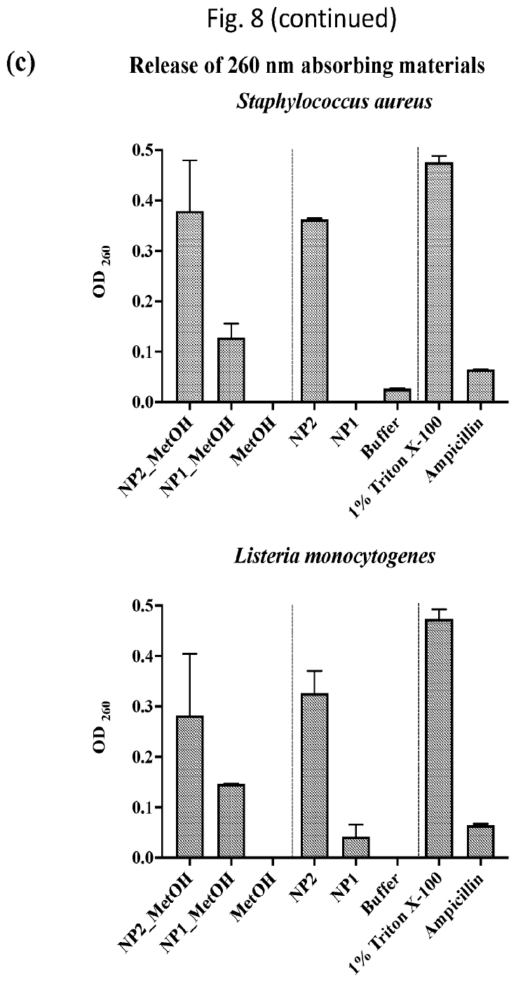

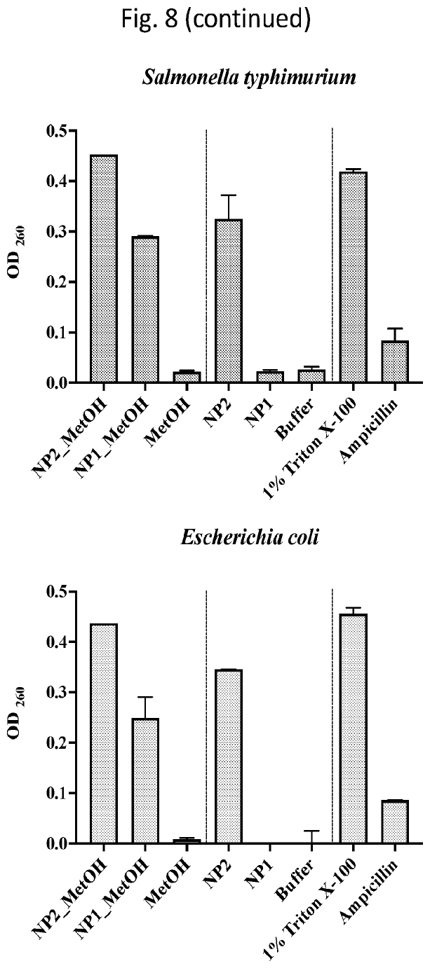

An increase in the fluorescent intensity of SYTOX® Green was observed in response to treatment with ethyl acetate extract of NP1 and NP2 for 6 hours as compared to control (methanol (MetOH)) (FIG. 8a). An increase in fluorescent intensity of N-phenyl-1-napthylamine (NPN) dye was also observed after this treatment as compared to control (FIG. 8b).

The release of intracellular components is a good indicator of membrane integrity. Small ions such as potassium and phosphate tend to leach out of cells first, followed by larger molecules such as DNA, RNA, and other materials. The long bars represent extensive cell rupture (death) and the release of cellular material. Treatment with ethyl acetate extract of NP1 or NP2 resulted in the release of intracellular components due to membrane disruption compared to control (FIG. 8c).

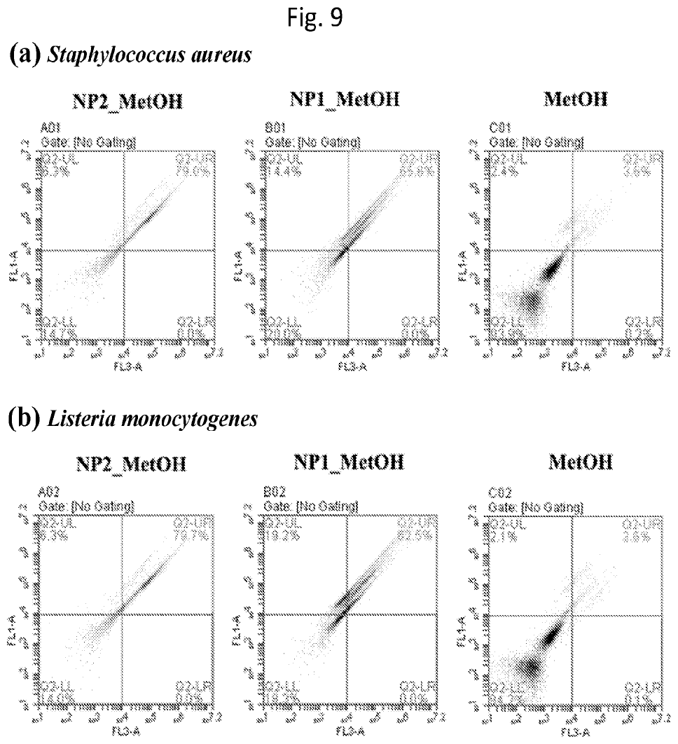

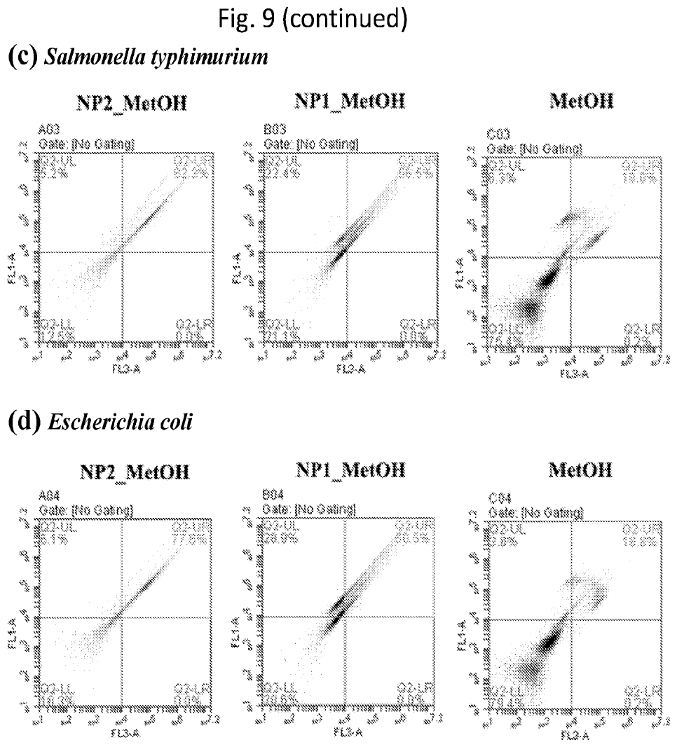

Additionally, flow cytometry with propidium iodide (PI) dye was used to determine whether treatment with ethyl acetate extract of NP1 or NP2 damaged cytoplasmic membranes. As is shown in FIG. 9, more than 70% of cells, of both Gram-positive and Gram-negative bacteria, exhibited high intensity PI fluorescence after being treated with NP2 ethyl acetate extract, and more than 50% of cells also exhibited high intensity PI fluorescence after being treated with NP1 ethyl acetate extract.

### Antifungal Activity of Fermentates

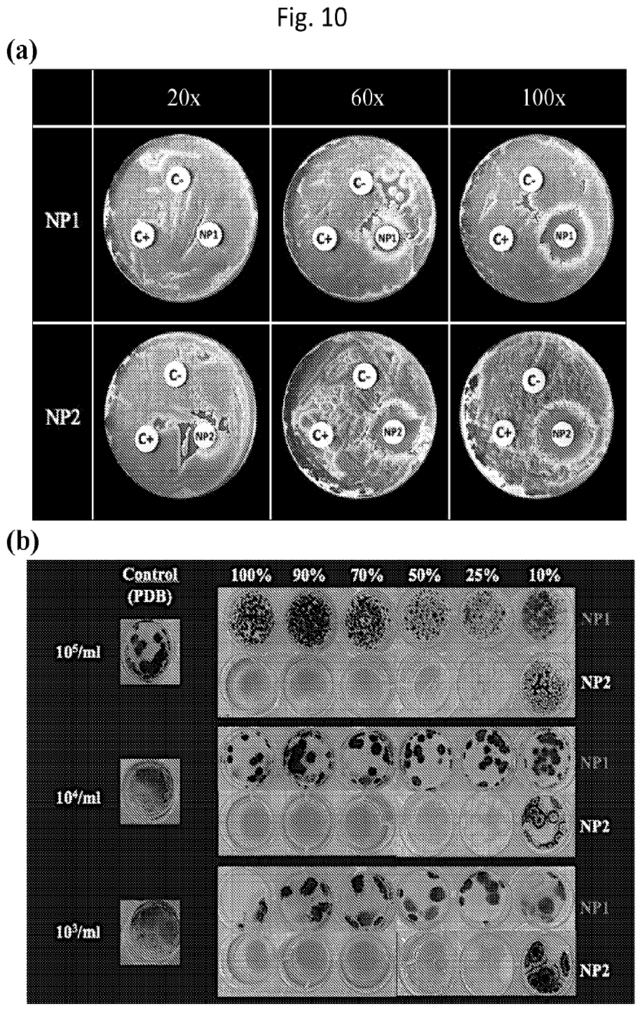

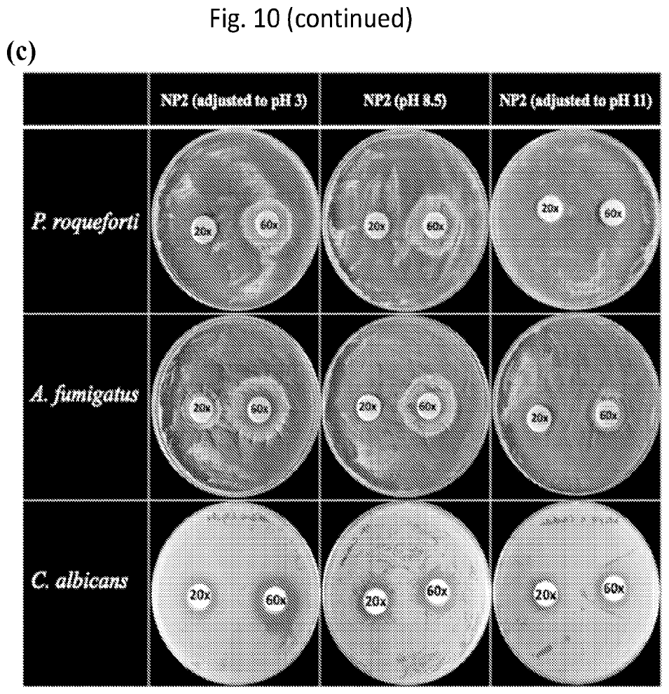

To determine antifungal activity of ethyl acetate extracts of NP1 and NP2, 20×, 60×, and 100× concentrated NP1 and NP2 ethyl acetate extracts were tested (FIG. 10a). Clear zones of inhibition were formed with 60× and 100× concentrated ethyl acetate extracts of NP1 and NP2 against P. roqueforti (FIG. 10a). Regardless of conidia inoculum size (10⁴/mL and 10³/mL), PDB liquid medium made in dilutions comprising at least 25% heat-treated NP2 as a solvent, inhibited the growth of A. fumigatus, while mold growth was observed in PDB liquid medium made in heat-treated NP1 (FIG. 10b). A 28.0±1.1 mm inhibition zone against P. roqueforti was produced using the 60× concentrated NP2 ethyl acetate extract adjusted to pH 3, whereas a 29.1±1.5 mm inhibition zone was produced with the original NP2 ethyl acetate extract, and no inhibition zone was produced with NP2 ethyl acetate extract adjusted to pH 11. The inhibition zones against A. fumigatus were 19.4±0.5 mm with 20× concentrated NP2 ethyl acetate extract at pH 3, 31.5±2.6 mm with 60× concentrated NP2 ethyl acetate extract at pH 3, 26.4±0.9 mm with the original NP2 ethyl acetate extract, and 17.3±2.6 mm with 60× concentrated NP2 ethyl acetate extract at pH 11 (FIG. 10c). Thus, NP2 consistently displays high inhibitory activity across a wide pH range.

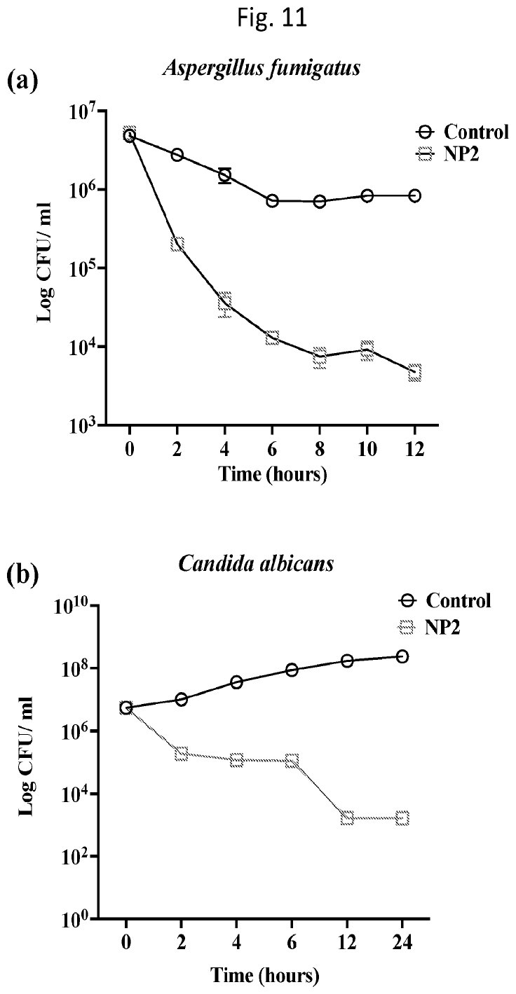

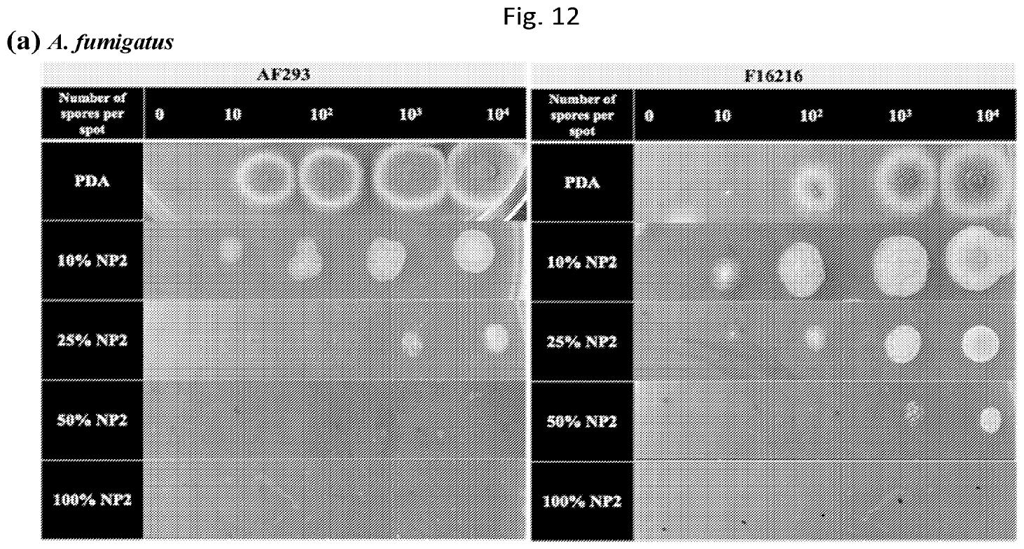

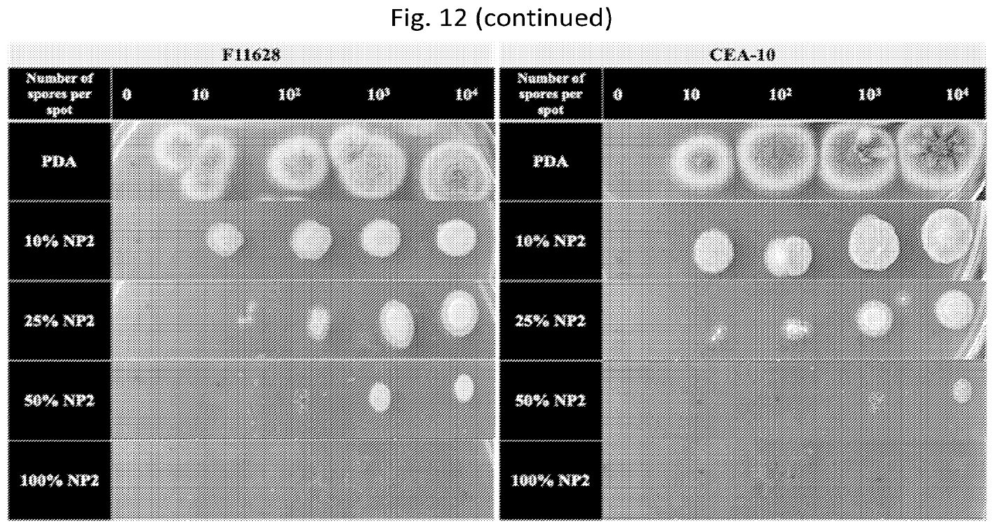

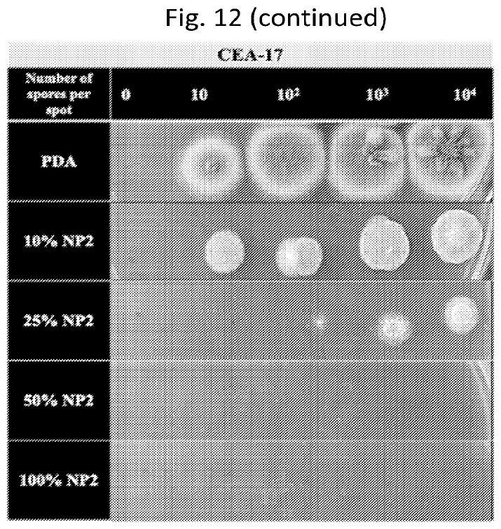

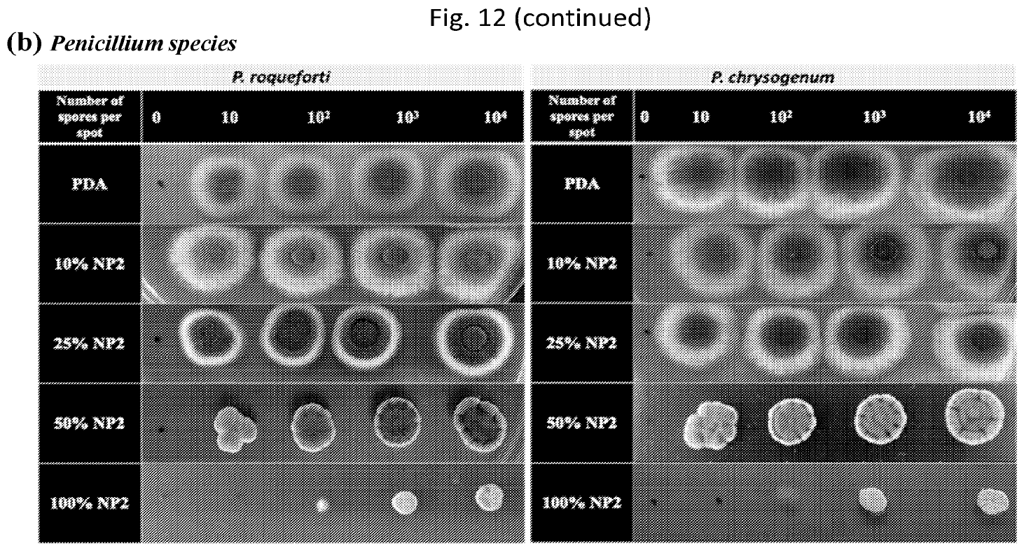

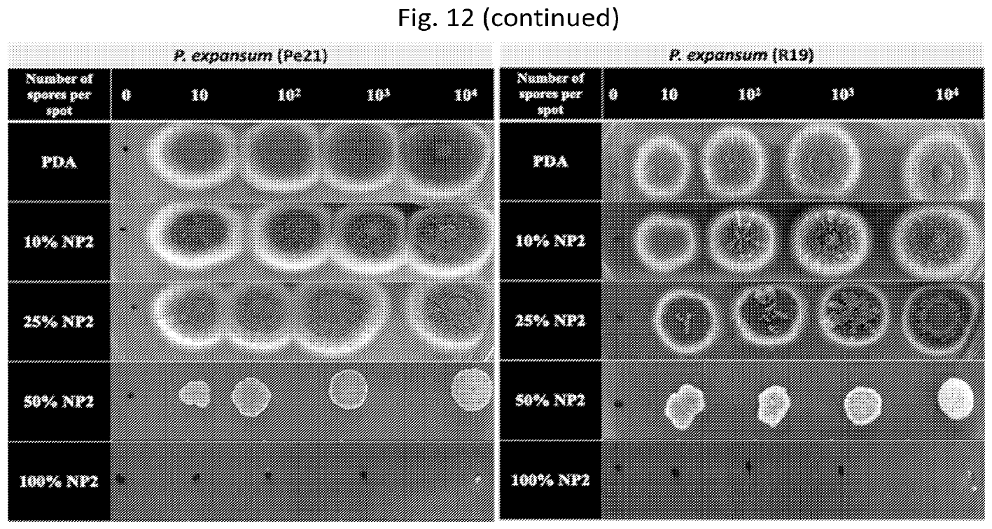

Treatment with NP2 ethyl acetate extract resulted in a 10,000-fold reduction of A. fumigatus spores and 1,000-fold reduction of C. albicans cells in 12 hours, indicating that NP2 can kill 99.99% of A. fumigatus spores (FIG. 11a) and can kill 99.9% of C. albicans (FIG. 11b). 10 μL of spore suspensions of five different strains of A. fumigatus (AF293, F16216, F11628, CEA-10, CEA-17) (FIG. 12a) and three different Penicillium species (P. roqueforti, P. chrysogemum, and P. expansum) (FIG. 12b) were spot plated onto solid PDA medium generated using various dilutions of heat-treated NP2 as a solvent. While PDA with 100% NP2 shows near-complete blockage Penicillium growth, PDA containing as low as 10% NP2 displays high inhibition of A. fumigatus growth, indicating that NP2 effectively controls this opportunistic pathogenic fungus.

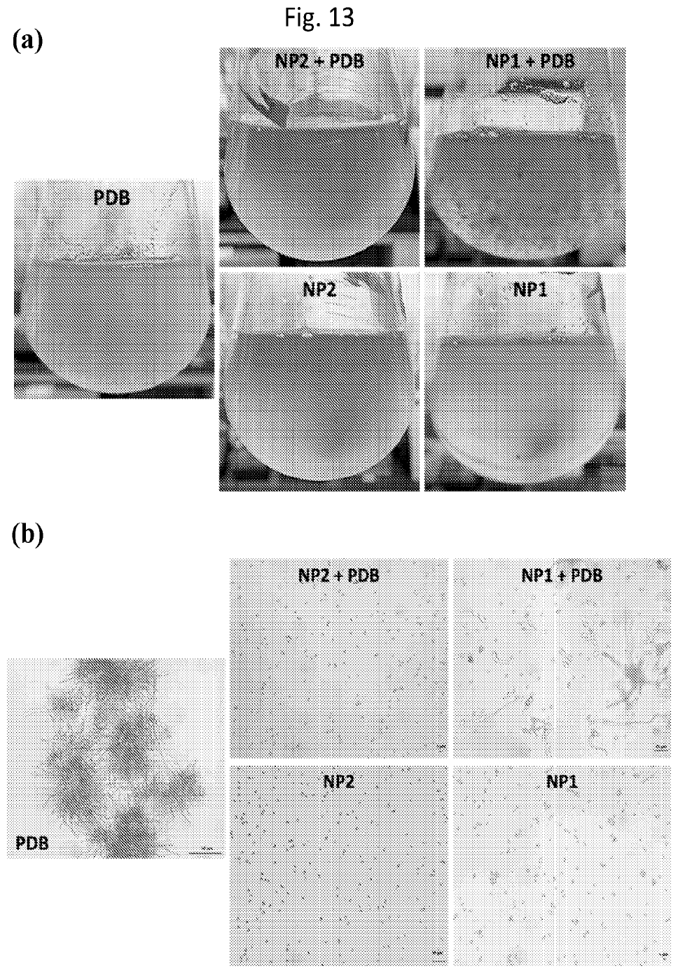

To test the antifungal activity of NP1 and NP2 fermentate against spores of A. fumigatus, 10⁵ conidia/mL of A. fumigatus was inoculated into five different liquid media: PDB (negative control), NP2, NP1, PDB made in 100% NP2 (NP2+PDB), and PDB made in 100% NP1 (NP1+PDB). While NP1 showed inhibition of spore germination and growth to a lesser degree, NP2 completely blocked spore germination, even in the presence of PDB (FIG. 13a-b).

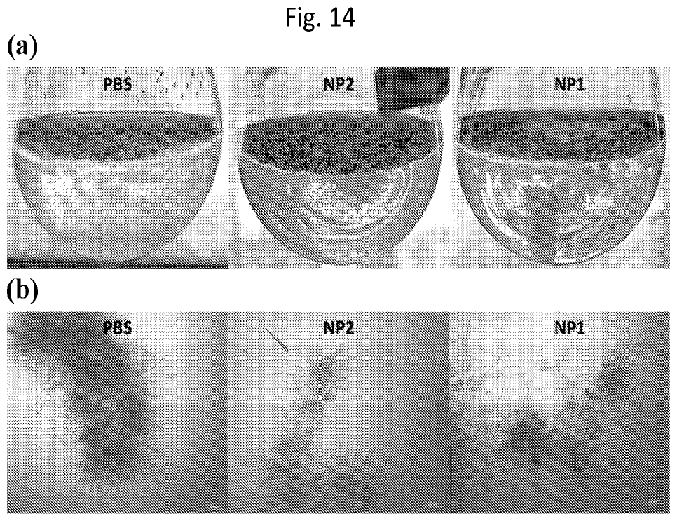

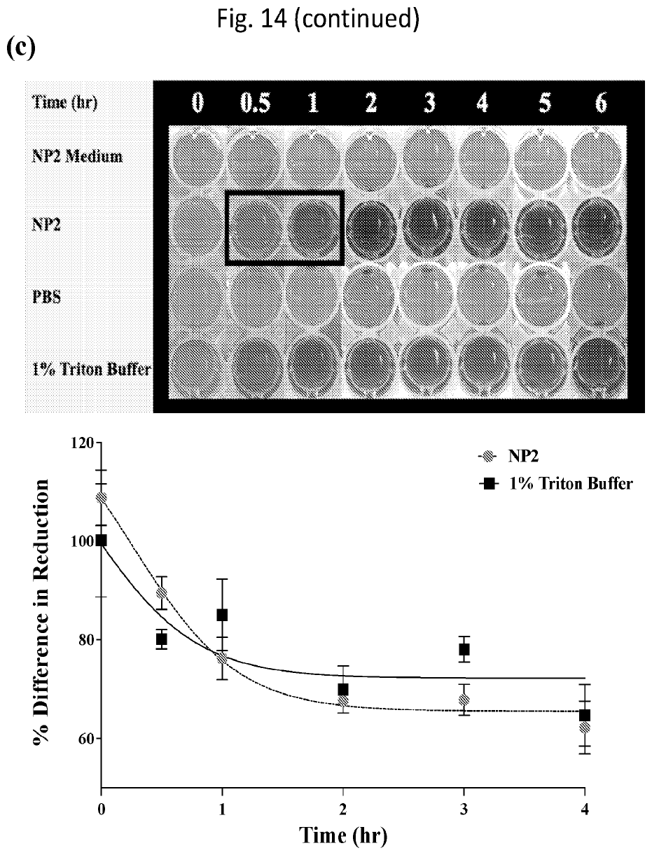

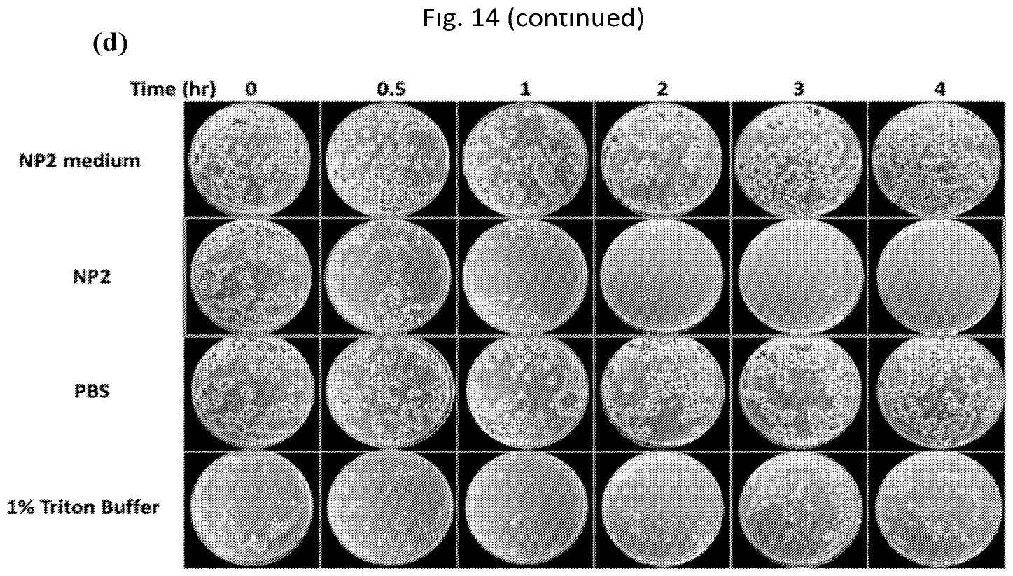

To test the antifungal activity of NP1 and NP2 on growing vegetative cells (mycelia), two grams of collected mycelia were reinoculated into three different flasks containing PBS (negative control), NP2, or NP1. Aggregated hyphal pellets were found in NP2 due to the inhibition of hyphal growth, but inhibition was not observed in PBS (control) and NP1 (FIG. 14a and FIG. 14b). The fungicidal activity of NP2 against vegetative cells of A. fumigatus was determined by Alamar Blue assay. Cell viability was assessed at 0, 0.5, 1, 2, 3, 4, 5, and 6 hours after inoculation in NP2 medium (negative control), NP2, PBS (negative control), and 1% Triton buffer (positive control). The growth of vegetative cells of A. fumigatus is inhibited by 38% in NP2 compared to that of the control after 4 hours of treatment (FIG. 14c). Fungal cells treated with NP2 for 1 hour or longer did not proliferate, indicating cell death caused by NP2 antimicrobial compound(s) (FIG. 14d).

### Mechanism of the Fungicidal Activity

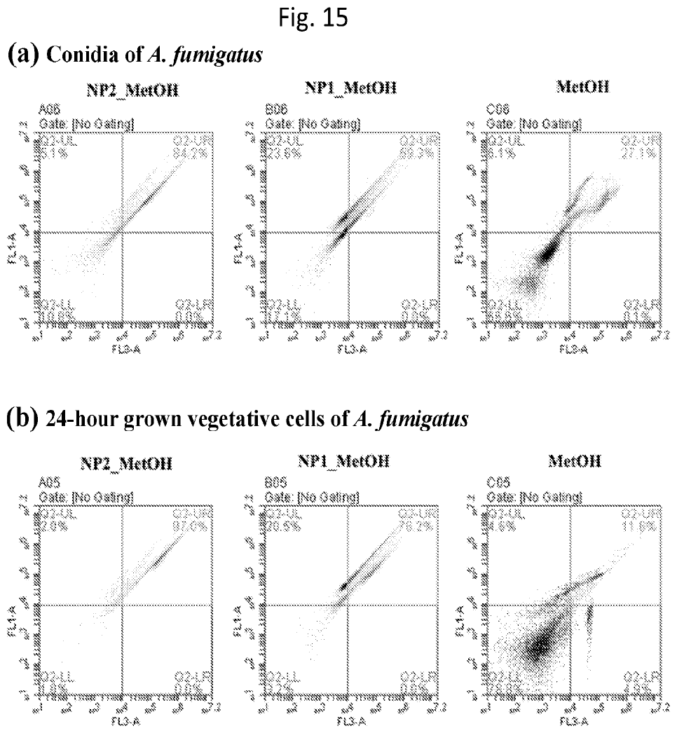

Cell membrane permeability was assessed via flow cytometry as described above. More than 84% of conidia (FIG. 15a) and 97% of vegetative cells (FIG. 15b) of A. fumigatus exhibited high intensity PI fluorescence after being treated with NP2 ethyl acetate extract, and 59% of conidia and 76% of vegetative cells of A. fumigatus exhibited high intensity PI fluorescence after being treated with NP1 ethyl acetate extract. This indicates that vegetative cells were more susceptible to the fermentate extracts than conidia.

### Inhibition of Mold Growth on Cheeses

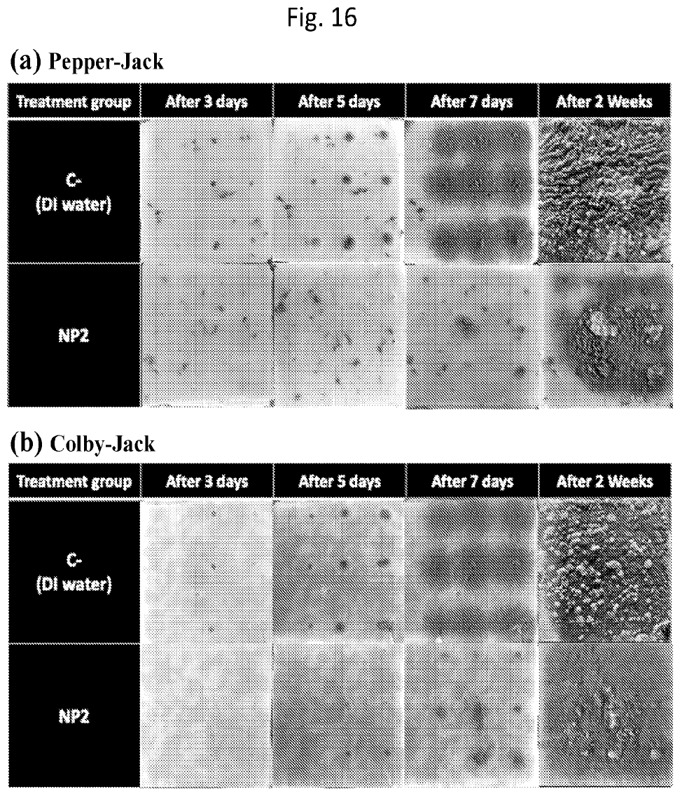

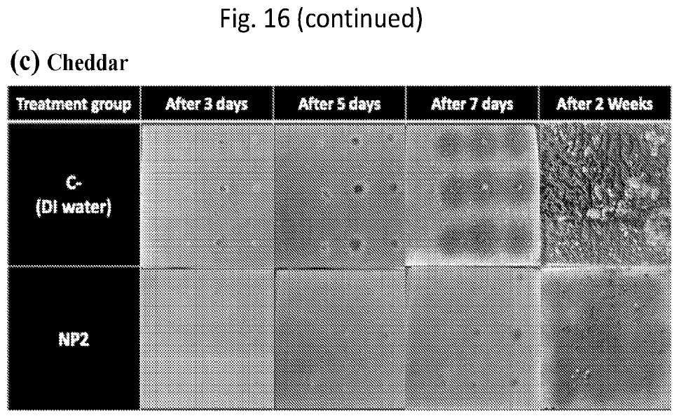

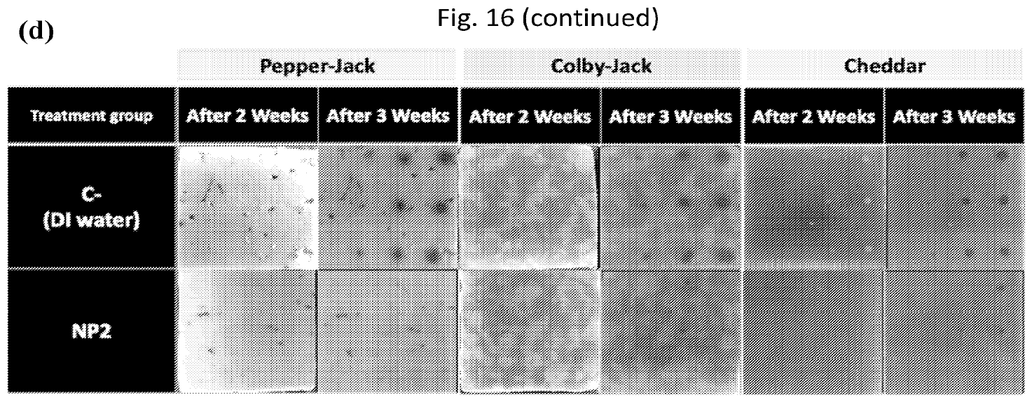

Three different types of commercially available sliced cheeses (i.e., pepper jack, Colby jack, and cheddar) were dipped into DI water or NP2, both containing 0.5% xanthan gum, for 5 seconds. While mold growth was visible at 3 days, 5 days, 1 week, and 2 weeks of incubation in the control groups, it was effectively inhibited in the NP2-treated cheeses (FIG. 16a-c). Additionally, the above-mentioned cheeses were incubated at 4° C. for 3 weeks and mold growth was checked after 2 weeks and 3 weeks of cold-room storage. Again, the NP2-treated cheeses showed effective blockage of mold growth (FIG. 16d). Thus, NP2 can be used as a food grade antifungal agent.

### Antimicrobial Activity of Fermentates Made in Combinations of NP1 Medium and NP2 Medium

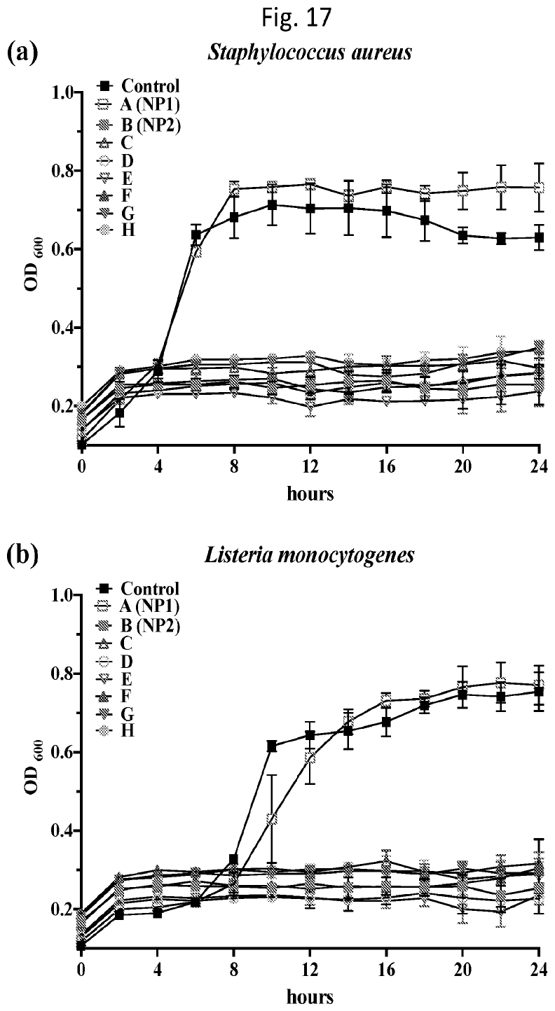

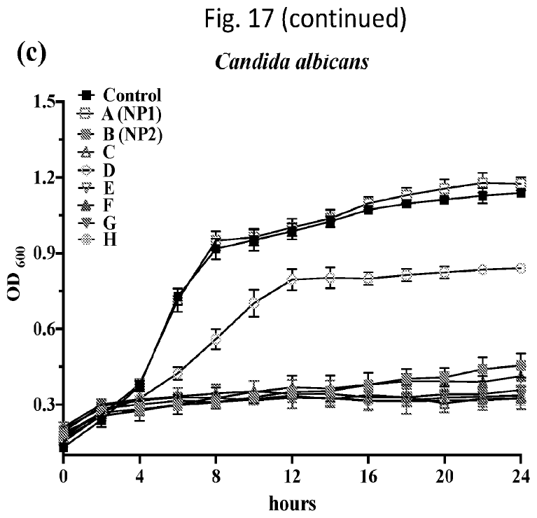

TSB liquid medium and YPD liquid medium were made using fermentates obtained by growing A. oryzae in culture broths composed of various ratios of NP1/NP2 media components (Table 5) as a solvent. All combination NP1/NP2 media show strong antibacterial activities against S. aureus (FIG. 17a) and L. monocytogenes (FIG. 17b). All combination NP1/NP2 media except for 90% NP1/10% NP2 inhibited the growth of C. albicans (FIG. 17c). Fungal fermentates prepared in medium comprising as little as 3 g of NP2 components per liter show highly heat stable antibacterial and antifungal activity.

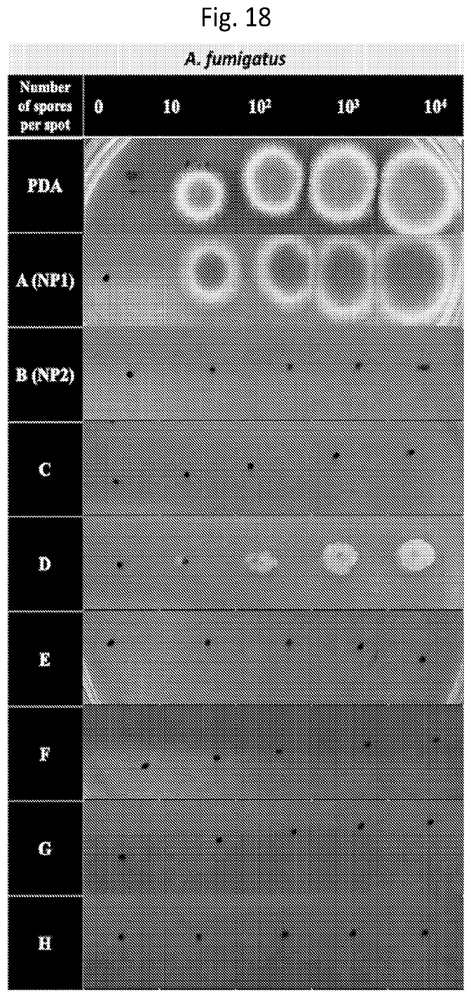

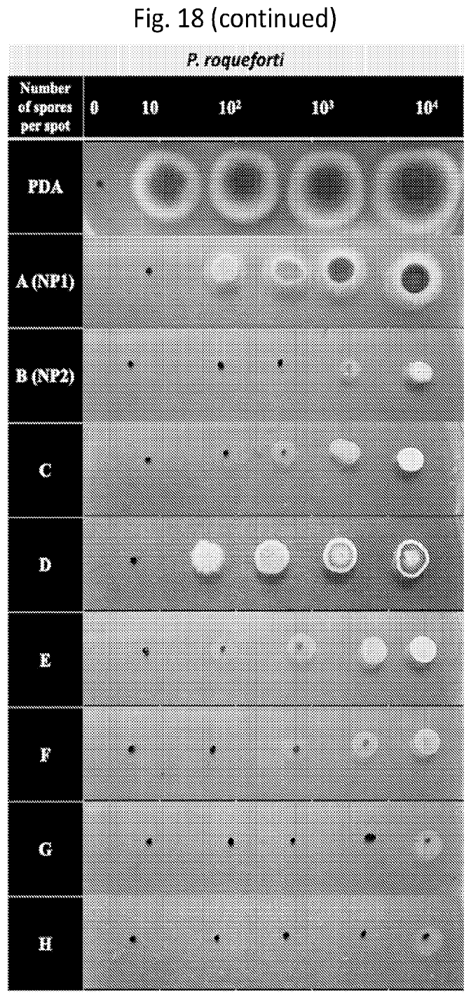

The culture medium made using fungal fermentates obtained by growing A. oryzae in culture broths having 10-25% NP1 medium components and 75-90% NP2 medium components (see G and H in Table 5) display enhanced antifungal activity compared to NP1 (A) and NP2 (B) alone (FIG. 18).

### Pancreatic Digest of Casein is the Essential Component of NP2 Medium

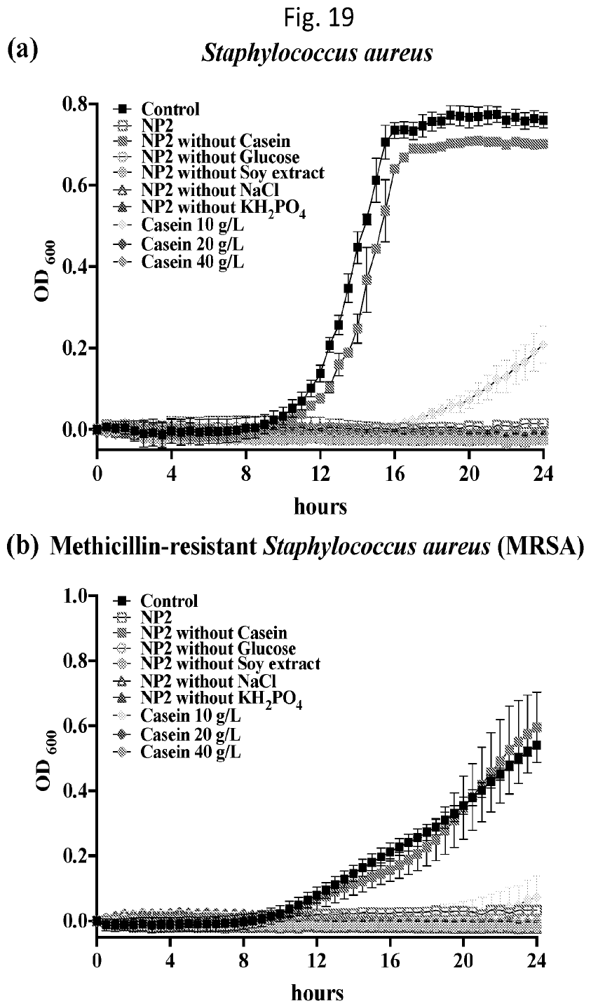

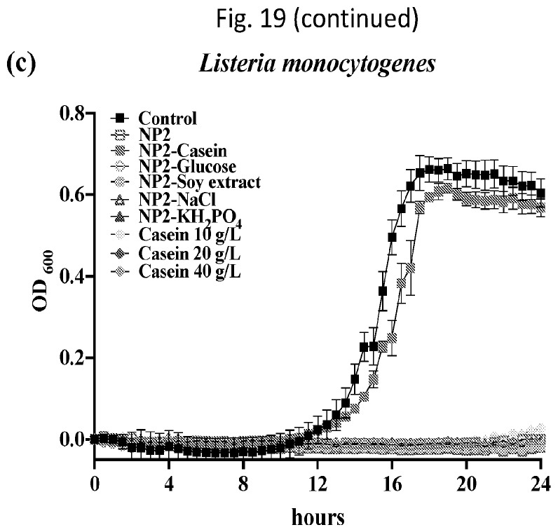

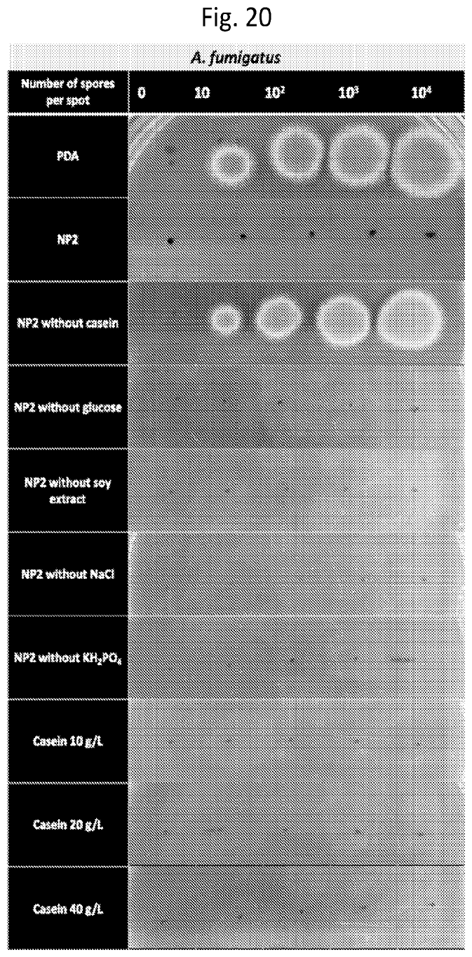

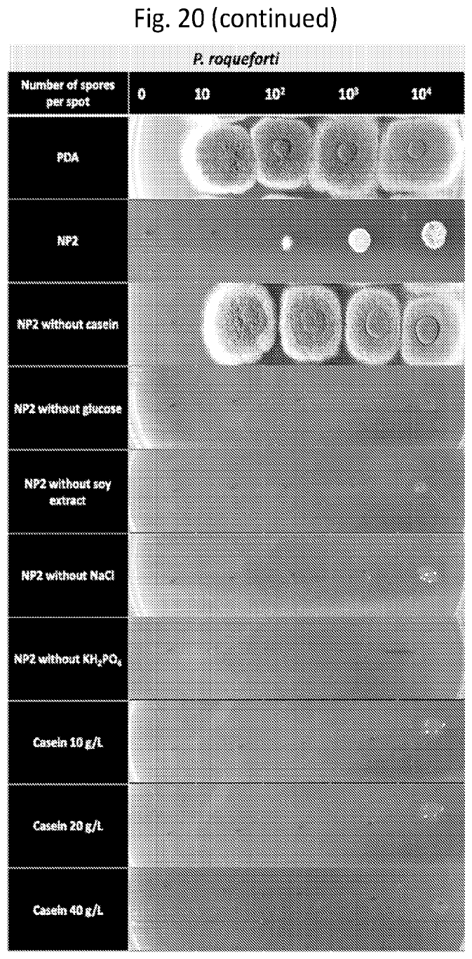

While TSB liquid medium generated using heat-treated fermentate prepared in NP2 medium lacking pancreatic digest of casein did not inhibit the growth of S. aureus (FIG. 19a) methicillin-resistant S. aureus (MRSA) (FIG. 19b), and L. monocytogenes (FIG. 19c), TSB liquid medium generated using heat-treated fermentate prepared in medium comprising only 20-40 g/L of pancreatic digest of casein prevented the growth of all three of these bacteria. Thus, pancreatic digest of casein is the essential medium component required for the antimicrobial activity of NP2. Similar results were obtained with A. fumigatus and P. roqueforti. As shown in FIG. 20, when A. fumigatus and P. roqueforti were inoculated at conidial counts of 0, 10, 100, 1,000, 10,000 per spot on PDA solid medium made using the heat-treated fungal fermentates as described in FIG. 19 as a solvent, the modified NP2 fermentates and the fermentates produced in casein (10-40 g/L) only media show at least as much or even enhanced antifungal activity against A. fumigatus and P. roqueforti.

1. A method of producing an antimicrobial composition, the method comprising:
a) inoculating a medium with an Aspergillus sp. to produce an inoculated medium;
b) culturing the inoculated medium to produce an Aspergillus fermentate;
c) filtering the Aspergillus fermentate to obtain a cell-free Aspergillus fermentate filtrate.

2. The method of claim 1, further comprising:
d) extracting the cell-free Aspergillus fermentate filtrate to obtain an extract of the cell-free Aspergillus fermentate filtrate.

3. The method of claim 2, wherein the extraction is performed using an extractant.

4. The method of claim 2, further comprising:
e) concentrating the extract to obtain an extract concentrate.

5. The method of claim 4, further comprising:
f) reconstituting the extract concentrate with a reconstitution solvent.

6. The method of claim 5, wherein the reconstitution solvent comprises methanol.

7. The method of claim 1, wherein the medium comprises an enzymatic digest of casein.

8. The method of claim 1, wherein the medium comprises one or more of a malt extract, maltose, dextrose, a yeast extract, a papaic digest of soybean, dextrose, sodium chloride, dipotassium phosphate, or any combination thereof.

9. The method of claim 1, wherein the Aspergillus sp. is Aspergillus oryzae, Aspergillus terreus, Aspergillus sojae, Aspergillus nidulans, Aspergillus fumigatus, Aspergillus tubingensis, Aspergillus carbonarius, Aspergillus parasticus, or Aspergillus flavus.

10. The method of claim 1, wherein:
i) the medium is inoculated with about 10⁴ to 10⁷ conidia/mL of the Aspergillus sp.,
ii) the inoculated medium is cultured for about 6 to 10 days,
iii) the inoculated medium is cultured at about 22 to 30° C.,
iv) the inoculated medium is cultured with shaking at about 150 to 220 rpm, or
v) any combination thereof.

11. An antimicrobial composition produced by the method of claim 1.

12. The antimicrobial composition of claim 11, wherein the antimicrobial composition has antibacterial activity against one or more bacterium selected from Salmonella typhimurium, Escherichia coli, Staphylococcus aureus, and Listeria monocytogenes.

13. The antimicrobial composition of claim 11, wherein the antimicrobial composition has antifungal activity against one or more fungus selected from Aspergillus flavus, Aspergillus fumigatus, Candida albicans, Candida auris, Penicillium roqueforti, Penicillium chrysogemum, and Penicillium expansum.

14. The antimicrobial composition of claim 11, wherein the composition is food grade.

15. The antimicrobial composition of claim 14, further comprising a food-grade binding agent.

16. A food product comprising the antimicrobial composition of claim 14.

17. A medical device comprising the antimicrobial composition of claim 11.

18. A method of inhibiting the growth of a microbe in or on a food product, the method comprising: applying an effective amount of the antimicrobial composition of claim 14 to the food product.

19. A method of inhibiting the growth of a microbe on a surface, the method comprising: applying an effective amount of the antimicrobial composition of claim 11 to the surface.

20. A method of inhibiting the growth of a microbe in or on a subject, the method comprising: administering an effective amount of the antimicrobial composition of claim 11 to the subject.
